(19) United States
(12) Patent Application Publication
(10) Pub. No.: US 2024/0417447 A1
(43) Pub. Date: Dec. 19, 2024

(54) DOSAGE REGIMENS FOR AND COMPOSITIONS INCLUDING ANTI-RSV ANTIBODIES

(71) Applicants: MEDIMMUNE LIMITED, Cambridge (GB); SANOFI PASTEUR INC., Swiftwater, PA (US)

(72) Inventors: Anis Ahmed Khan, Gaithersburg, MD (US); Vadryn Pierre, Gaithersburg, MD (US)

(21) Appl. No.: 18/669,241

(22) Filed: May 20, 2024

Related U.S. Application Data
(62) Division of application No. 16/859,750, filed on Apr. 27, 2020, now Pat. No. 12,024,553.
(60) Provisional application No. 62/840,701, filed on Apr. 30, 2019.

(51) Int. Cl. C07K 16/10; A61K 39/00; A61K 47/18; A61K 47/26; A61P 31/14

(52) U.S. Cl. CPC C07K 16/1027; A61K 47/183; A61K 47/26; A61P 31/14; A61K 2039/545; C07K 2317/21; C07K 2317/76

(57) ABSTRACT

In one aspect, this disclosure describes methods of treating or preventing RSV infection in a patient in need thereof. The methods including dosing regiments for administering a composition including a fixed dose of an anti-RSV monoclonal antibody or an antigen binding fragment thereof. In another aspect, this disclosure describes pharmaceutical compositions for the treatment or prevention of RSV infection. In yet another aspect, this disclosure describes a pharmaceutical unit dose including nirsevimab.

### RELATED APPLICATIONS

This application claims the benefit of U.S. Provisional Application Ser. No. 62/840,701,filed Apr. 30, 2019, which is incorporated by reference herein.

### SEQUENCE LISTING

This application contains a Sequence Listing electronically submitted to the United States Patent and Trademark Office via EFS-Web as an ASCII text file entitled “0490-000006US01_ST25.txt” having a size of 24 kilobytes and created on Apr. 27, 2020. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### BACKGROUND

Respiratory syncytial virus (RSV) is the most common cause of lower respiratory tract infections among infants and children worldwide. Nearly all children are infected with RSV during the first 2 years of life. In 2005, RSV was responsible for more than 30 million episodes of new lower respiratory tract infections among children 5 years and younger, resulting in an estimated 66,000-199,000 deaths globally.

While all children are at risk for severe lower respiratory tract infections during their primary infection, healthy term infants 3 months of age and younger account for more RSV-associated hospitalizations than any other group. Severe illness during infancy has the potential to cause both acute and long-term pulmonary sequelae, including recurrent wheezing episodes throughout childhood.

Currently, the only approved prophylaxis for RSV disease is palivizumab (SYNAGIS; MedImmune, Gaithersburg, MD). Palivizumab is an RSV fusion (F)-specific immunoglobulin G monoclonal antibody indicated for the prevention of serious lower respiratory tract disease caused by RSV in children at high risk, including preterm infants born at 35 weeks gestational age or less. Due in part to the high cost of palivizumab prophylaxis, the most recent guidance from the American Academy of Pediatrics does not recommend it for healthy preterm infants born at or after 29 weeks gestational age. Moreover, safe and effective active vaccines remain elusive. Additional means for RSV prophylaxis, particularly for use in healthy term infants, would therefore be advantageous.

### SUMMARY OF THE INVENTION

This disclosure describes methods, including a dosing regimen for an anti-RSV antibody or fragment thereof; pharmaceutical compositions; and pharmaceutical unit doses for the treatment or prevention of RSV infection in a patient.

In one aspect, this disclosure describes methods of treating or preventing RSV infection in a patient in need thereof.

In some embodiments, a method of treating or preventing RSV infection in a patient in need thereof includes determining the age of the patient. In response to the patient being in the second year of life, the method includes administering a composition comprising a fixed dose of 200 milligrams (mg) of an anti-RSV monoclonal antibody or an antigen binding fragment thereof. In response to the patient being in the first year of life, the method includes determining the patient's weight and, in response to the patient having a weight of at least 5 kilograms (kg), the method includes administering a composition comprising a fixed dose of 100 mg of an anti-RSV monoclonal antibody or an antigen binding fragment thereof.

In some embodiments, a method of treating or preventing RSV infection in a patient in need thereof includes administering a fixed dose of 200 mg of an anti-RSV monoclonal antibody or an antigen binding fragment thereof to a patient having a weight of at least 5 kg.

In some embodiments, a method of treating or preventing RSV infection in a patient in need thereof includes determining the weight of a patient and administering to a patient having a weight of at least 5 kg a fixed dose of 200 mg of an anti-RSV monoclonal antibody or an antigen binding fragment thereof or administering to a patient having a weight of up to 5 kg a fixed dose of 100 mg of an anti-RSV monoclonal antibody or an antigen binding fragment thereof.

In some embodiments, a patient having a weight of at least 5 kg has a weight of up to 10 kg, up to 15 kg, or up to 20 kg.

In some embodiments, a method of treating or preventing RSV infection in a patient in need thereof includes determining the age of the patient and administering a fixed dose of 200 mg of an anti-RSV monoclonal antibody or an antigen binding fragment thereof to a patient aged 3 months or older. In some embodiments, a method of treating or preventing RSV infection in a patient in need thereof includes determining the age of the patient and administering a fixed dose of 200 mg of an anti-RSV monoclonal antibody or an antigen binding fragment thereof to a patient aged 6 months or older.

In some embodiments, a method of treating or preventing RSV infection in a patient in need thereof includes determining the age of the patient and administering a fixed dose of 200 mg of an anti-RSV monoclonal antibody or an antigen binding fragment thereof to a patient aged 3 months or older and administering a fixed dose of 100 mg of an anti-RSV monoclonal antibody or an antigen binding fragment thereof to a patient aged up to 3 months.

In some embodiments, a method of treating or preventing RSV infection in a patient in need thereof includes determining the age of the patient and administering a fixed dose of 200 mg of an anti-RSV monoclonal antibody or an antigen binding fragment thereof to a patient aged 6 months or older and administering a fixed dose of 100 mg of an anti-RSV monoclonal antibody or an antigen binding fragment thereof to a patient aged up to 6 months.

In some embodiments, a method of treating or preventing RSV infection in a patient in need thereof includes determining whether the patient is experiencing their first or second RSV season. In response to the patient experiencing their second RSV season, the method includes administering a composition comprising a fixed dose of 200 milligrams (mg) of an anti-RSV monoclonal antibody or an antigen binding fragment thereof. In response to the patient experiencing their first RSV season, the method includes determining the patient's weight; and in response to the patient having a weight of at least 5 kilograms (kg), administering a composition comprising a fixed dose of 100 mg of an anti-RSV monoclonal antibody or an antigen binding fragment thereof.

In some embodiments, a method of treating or preventing RSV infection in a patient in need thereof includes determining whether the patient is experiencing their first or second RSV season; and administering a fixed dose of 200 milligrams (mg) of an anti-RSV monoclonal antibody or an antigen binding fragment thereof to a patient experiencing their second RSV season.

In some embodiments, a method of treating or preventing RSV infection in a patient in need thereof includes determining whether the patient is experiencing their first or second RSV season. When the patient is experiencing their second RSV season, the method includes administering a fixed dose of 200 milligrams (mg) of an anti-RSV monoclonal antibody or an antigen binding fragment thereof to a patient. When the patient is experiencing their first RSV season, the method includes administering a fixed dose of 100 mg of an anti-RSV monoclonal antibody or an antigen binding fragment thereof to a patient, wherein the patient is experiencing their first RSV season.

In some embodiments, the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320, or both.

In some embodiments, the anti-RSV monoclonal antibody includes nirsevimab. In some embodiments, the antigen binding fragment of the anti-RSV monoclonal antibody includes an antigen binding fragment of nirsevimab.

In some embodiments, the method may include administering an anti-RSV monoclonal antibody or an antigen binding fragment thereof in a composition. In some embodiments, the composition includes an ionic excipient, a buffer, a sugar, and/or a surfactant. In some embodiments, the ionic excipient includes L-arginine hydrochloride at a concentration of 80 mM. In some embodiments, buffer includes 30 mM L-histidine/L-histidine hydrochloride. In some embodiments, the sugar includes 120 mM sucrose. In some embodiments, the surfactant includes polysorbate-80 in a range of 0.01% (w/v) to 0.05% (w/v). In some embodiments, the composition may have a pH in a range of from 5.5 to 6.5. In some embodiments, the anti-RSV monoclonal antibody or an antigen binding fragment thereof may be present in the composition at a concentration of 100 mg/mL.

In another aspect, this disclosure describes pharmaceutical compositions for the treatment or prevention of RSV infection. In some embodiments, the pharmaceutical compositions may include 100 mg of nirsevimab, and the composition may be administered to a patient in the first year of life, the patient having a weight of at least 5 kg. In some embodiments, the pharmaceutical compositions may include 200 mg of nirsevimab, and the composition may be administered to a patient in the second year of life.

In some embodiments, after administration of the pharmaceutical composition, the patient exhibits AUC_{0-∞} of greater than 13.4 day·mg/mL.

In some embodiments, the pharmaceutical composition includes an ionic excipient, a buffer, a sugar, and/or a surfactant. In some embodiments, the ionic excipient includes L-arginine hydrochloride at a concentration of 80 mM. In some embodiments, buffer includes 30 mM L-histidine/L-histidine hydrochloride. In some embodiments, the sugar includes 120 mM sucrose. In some embodiments, the surfactant includes polysorbate-80 in a range of 0.01% (w/v) to 0.05% (w/v). In some embodiments, the composition may have a pH in a range of from 5.5 to 6.5. In some embodiments, the nirsevimab thereof may be present in the composition at a concentration of 100 mg/mL.

In yet another aspect, this disclosure describes a pharmaceutical unit dose including nirsevimab. In some embodiments, the pharmaceutical unit dose includes 100 mg of nirsevimab. In some embodiments, the pharmaceutical unit dose includes 200 mg of nirsevimab. In some embodiments, the unit dose is suitable for intramuscular administration.

In some embodiments, the pharmaceutical unit dose comprises a composition comprising nirsevimab. In some embodiments, the composition includes an ionic excipient, a buffer, a sugar, and/or a surfactant. In some embodiments, the ionic excipient includes L-arginine hydrochloride at a concentration of 80 mM. In some embodiments, buffer includes 30 mM L-histidine/L-histidine hydrochloride. In some embodiments, the sugar includes 120 mM sucrose. In some embodiments, the surfactant includes polysorbate-80 in a range of 0.01% (w/v) to 0.05% (w/v). In some embodiments, the composition may have a pH in a range of from 5.5 to 6.5. In some embodiments, the nirsevimab thereof may be present in the composition at a concentration of 100 mg/mL.

In some embodiments, the nirsevimab in the composition is stable at 2° C. to 8° C. for at least 3 months as determined by high performance size exclusion chromatography (HPSEC).

As used herein, the terms “antibody” or “immunoglobulin” refer to a tetrameric glycoprotein that consists of two heavy chains and two light chains, each comprising a variable region and a constant region. Antigen-binding portions may be produced by recombinant DNA techniques or by enzymatic or chemical cleavage of intact antibodies. The term “antibody” includes monoclonal antibodies, polyclonal antibodies, chimeric antibodies, human antibodies, and humanized antibodies.

As used herein, the terms “antibody” or “antigen binding fragment thereof” include man-made antibodies such as monoclonal antibodies (mAbs) and/or an antigen binding fragments thereof, produced by conventional hybridoma technology, by phage display, and/or recombinant technology. The terms include both intact immunoglobulin molecules including, for example, a polyclonal antibody, a monoclonal antibody (mAb), a monospecific antibody, a bispecific antibody, a polyspecific antibody, as well as portions, fragments, regions, peptides and derivatives thereof (provided by any known technique, such as, but not limited to, enzymatic cleavage, peptide synthesis, or recombinant techniques), such as, for example, immunoglobulin devoid of light chains, Fab, Fab′, F(ab′)₂, Fv, scFv, antibody fragment, diabody, Fd, CDR regions, or any portion or peptide sequence of the antibody that is capable of binding antigen or epitope. The antibody, or antigen binding fragment thereof, may be a human antibody, a humanized antibody, an animal antibody (e.g. camelid antibody), or chimeric antibody. In one embodiment, the “antigen binding fragment thereof” is a single chain antibody, a single chain variable fragment (scFv), a Fab fragment, or a F(ab′)₂ fragment.

As used herein, the term “monoclonal antibody” refers to an antibody obtained from a population of substantially homogeneous antibodies, that is, the individual antibodies comprising the population are identical except for possible naturally occurring mutations that may be present in minor amounts.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

### BRIEF DESCRIPTION OF THE FIGURES

FIG. 1A-FIG. 1B show nirsevimab light chain nucleotide sequence (SEQ ID NO:11) and translation (SEQ ID NO:1). CDRs are underlined, and the division between the variable and constant regions is marked by a ‘|’.

FIG. 2A-FIG. 2C show nirsevimab heavy chain nucleotide sequence (SEQ ID NO:12) and translation (SEQ ID NO:2). CDRs are underlined, and the division between the variable and constant regions is marked by a ‘|’. Locations of three amino acid substitutions (M252Y/S254T/T256E; “YTE”) in the CH2 region of the Fc domain, introduced to increase the serum half-life of nirsevimab, are circled.

FIG. 3 shows a schematic of the Phase 2b study design, as further described in Example 1. ADA=anti-drug antibody; IM=intramuscular; LRTI=lower respiratory tract infection; PK=pharmacokinetics. PK and ADA samples were collected during screening; on Days 91, 151, 361; and at hospitalisation for LRTI. Safety assessments were performed from screening through Day 361. * Screening and Day 1 visits could occur on the same day.

FIG. 4 shows incidence of all medically attended lower respiratory tract infections through 150 days post dose, as further described in Example 1. LRTI=lower respiratory tract infection; RSV=respiratory syncytial virus.

FIG. 5 shows a Kaplan-Meier plot for time to first medically attended RSV-confirmed LRTI through 150 days post dose, as further described in Example 1. LRTI=lower respiratory tract infection; No=number; RSV=respiratory syncytial virus. P-values were obtained from stratified log-rank test and Wilcoxon test with 2 stratification factors (age at randomisation and hemisphere) as the strata.

FIG. 6A-FIG. 6B show a Forest plot for subgroup analysis for incidence of medically attended RSV-confirmed LRTI (observed) through 150 days post dose, as further described in Example 1. CI=confidence interval; LRTI=lower respiratory tract infection; RRR=relative risk reduction; RSV=respiratory syncytial virus.

FIG. 7 shows a schematic of an exemplary population pharmacokinetic (popPK) analysis work flow, as further described in Example 2.

FIG. 8 shows a schematic of an exemplary population pharmacokinetic (popPK) structural model.

FIG. 9A shows postmenstrual age of infants exposed to nirsevimab in Phase 1b/2a and Phase 2b. FIG. 9B shows baseline bodyweight distribution of infants exposed to nirsevimab in Phase 1b/2a and Phase 2b.

FIG. 10 shows distribution of available PK Data by study. Study 1—Phase 1 study in healthy adult volunteers (Griffin et al. 2017, Antimicrob Agents Chemother. 61(3), pii: e01714-16); Study 2—the Phase 1b/2a study in healthy preterm infants 32 to <35 weeks gestational age (GA) (Domachowske et al. Pediatr Infect Dis J. 2018; 37(9):886-892); and Study 3—the Phase 2b study in healthy preterm infants 29 to <35 weeks GA (Example 1).

FIG. 11 shows effects of size and maturation on systemic clearance of nirsevimab.

FIG. 12 shows a goodness of fit plot: Observed versus Individual (left panel) and Observed versus Population (right panel) predicted nirsevimab concentrations.

FIG. 13A-FIG. 13C show diagnostic plots of conditional weighted residuals. FIG. 13A shows population predicted nirsevimab concentrations in mcg/mL. FIG. 13B shows time after dose in days. FIG. 13C shows density of the conditional weighted residuals.

FIG. 14 shows a visual predictive check of infant PK Data by scheduled visit.

FIG. 15 shows a Kaplan-Meier plot of medically attended respiratory syncytial virus-confirmed lower respiratory tract infection (MALRTI) outcome in Phase 2b study stratified by AUC quartiles. AUC_{0-∞}=area under the concentration-time curve from time 0 to infinity; MALRTI=medically attended respiratory syncytial virus-confirmed lower respiratory tract infection; Q=quartile. Figure presents data only from subjects in the As-treated Population who had at least one measurable post-dose nirsevimab serum concentration.

FIG. 16 shows a visual predictive check (VPC) of MALRTI by the geographical region, Northern in the left panel, Southern in the right panel. The indicated lines reflect the observed estimates for infants in the placebo group, those with serum AUC quartile 1, and AUC quartile 2, or greater. The area surrounding each line denotes the model-predicted 95% confidence interval around the median for each stratum.

FIG. 17 shows a Forest plot of covariate effects in the final hazard model.

FIG. 18 shows distribution of exposure quartiles across age and bodyweight. AUC_{0-∞}=area under the concentration-time curve from time 0 to infinity; Q=quartile. Figure presents data only from subjects in the As-treated Population who had at least one measurable post-dose nirsevimab serum concentration. The solid horizontal line at 5 kg and dashed vertical lines at 3 and 6 months highlight differences in Q1 distribution amongst various age and body weight ranges

FIG. 19 shows effects of body weight and age on exposure and efficacy. The efficacy of nirsevimab efficacy in subjects in the lowest AUC quartile did not differ from placebo; AUC ranges in Q2 to Q4 were the in-vivo clinical efficacy target.

FIG. 20 shows predicted AUC distribution of the proposed dose of nirsevimab in the more than 5 kg in Phase 2b population to match exposures as observed in up to 5 kg in the first RSV Season. AUC=area under the concentration-time curve. The numbers above x-axis refer to the number of subjects in each category. The projected AUC values are estimated as the ratio of the administered dose and systemic clearance for all infants as derived from the population-PK model estimates. The projected AUC distribution in pink highlights the projected exposure for the 360 infants from the Phase 2b population weighing ≥5 kg when they are dosed with 50 mg (studied dose) versus 100 mg (proposed) nirsevimab. The boxplot in yellow illustrates the AUC distribution for infants from the Phase 2b population weighing <5 kg. Figure presents data only from subjects in the As-treated Population who had at least one measurable post-dose nirsevimab serum concentration.

FIG. 21 shows distribution of age and bodyweight for virtual infants in Phase 3 and Phase 2/3 population in the 1^{st} RSV season. All virtual infants weighted 1.5 kg or >34 weeks gestational age (GA) at the time of the dose. Dose is a single fixed 50 mg IM dose or 100 mg IM dose. The boxes represent the interquartile range (25^{th} to 75^{th} percentile), the top and bottom error bars show the largest value within 1.5 times the 75^{th} or the 25^{th} percentile, respectively.

FIG. 22 shows predicted AUC for virtual infants in Phase 3 and Phase 2/3 Population in the 1^{st} RSV season. All virtual infants weighted 1.5 kg or >34 weeks gestational age (GA) at the time of the dose. Dose is a single fixed 50 mg IM dose or 100 mg IM dose. The dotted line reflects the target AUC threshold of 13.4 day·mg/mL. The boxes represent the interquartile range (25^{th} to 75^{th} percentile), the top and bottom error bars show the largest value within 1.5 times the 75^{th} or the 25^{th} percentile, respectively.

FIG. 23 shows distribution of age and bodyweight for virtual infants in Phase 2/3 population in the 2^{nd} RSV season. Dose is a single fixed 200 mg IM dose. The boxes represent the interquartile range (25^{th} to 75^{th} percentile), the top and bottom error bars show the largest value within 1.5 times the 75^{th} or the 25^{th} percentile, respectively.

FIG. 24 shows predicted AUC for virtual infants in Phase 2/3 population in the 2^{nd} RSV season. Dose is a single fixed 200 mg IM dose. The dotted line reflects the target AUC threshold of 13.4 day·mg/mL. The boxes represent the interquartile range (25^{th} to 75^{th} percentile), the top and bottom error bars show the largest value within 1.5 times the 75^{th} or the 25^{th} percentile, respectively.

FIG. 25 shows in vitro neutralization of RSV A2 (left panel) and RSVB 9320 (right panel) by various antibodies including nirsevimab (1G7), 1F5, 2D10, and D25 each inhibited RSV A2 and RSV B9320 as measured in a microneutralization assay.

FIG. 26 shows in vitro neutralization of RSV A2 (left panel) and RSVB 9320 (right panel) by various antibodies including D25, nirsevimab (1G7), and variants of nirsevimab (1G7-GLM, B12-1, E3-5, and E9-2) as measured in a microneutralization assay (c+v=cells plus virus).

FIG. 27A-FIG. 27B show the effect of birth month on an exemplary patient's age during an RSV season in the United States. FIG. 27A shows that a patient born in April (indicated by an unfilled arrow) may be experiencing their second RSV season in October of the year of their birth (while ˜7 months old, indicated by a filled arrow). FIG. 27B shows a patient born in June (indicated by an unfilled arrow) may at ˜7 months old (indicated by a filled arrow) be experiencing their first RSV season.

### DETAILED DESCRIPTION OF ILLUSTRATIVE EMBODIMENTS

This disclosure describes methods, including a dosing regimen for an anti-RSV antibody or fragment thereof; pharmaceutical compositions; and pharmaceutical unit doses for the treatment or prevention of RSV infection in a patient. In some embodiments, the anti-RSV antibody or fragment thereof includes nirsevimab (also known as MEDI8897) or a fragment of nirsevimab.

In one aspect, this disclosure describes a dosing regimen for an anti-RSV antibody or an antigen binding fragment thereof. In particular, the dosing regimen provides fixed doses for patients based on age or weight or both. In some embodiments, the anti-RSV antibody or an antigen binding fragment thereof includes a monoclonal antibody or an antigen binding fragment of a monoclonal antibody.

In some embodiments, the anti-RSV antibody or an antigen binding fragment thereof has an IC50 value of up to 10 ng/ml in an in vitro neutralization assay. In an exemplary in vitro neutralization assay, HEp-2 cells are infected with RSV and the antibody or antigen binding fragment thereof. In some embodiments, the IC50 is at least 1 ng/ml, at least 2 ng/ml, at least 3 ng/ml, at least 4 ng/ml, at least 5 ng/ml, at least 6 ng/ml, at least 7 ng/ml, or at least 8 ng/ml for RSV A subtype including, for example, RSV A2. In some embodiments, the IC50 is up to 9 ng/ml, up to 8 ng/ml, up to 7 ng/ml, up to 6 ng/ml, up to 5 ng/ml, up to 4 ng/ml, up to 3 ng/ml, or up to 2 ng/ml for RSV subtype including, for example, RSV A2. In some embodiments, the IC50 is at least 1 ng/ml, at least 2 ng/ml, at least 3 ng/ml, at least 4 ng/ml, at least 5 ng/ml, at least 6 ng/ml, at least 7 ng/ml, or at least 8 ng/ml for RSV B subtype including, for example, RSV B9320. In some embodiments, the IC50 is up to 9 ng/ml, up to 8 ng/ml, up to 7 ng/ml, up to 6 ng/ml, up to 5 ng/ml, up to 4 ng/ml, up to 3 ng/ml, or up to 2 ng/ml for RSV subtype B including, for example RSV B9320. In one embodiment, the IC50 is measured in the in vitro neutralization assay is measured as described in Example 3 and/or 4, including, for example, for RSV A2 and/or RSV B9320.

In some embodiments, the anti-RSV antibody or an antigen binding fragment thereof includes a human antibody or antigen binding fragment thereof. The use of human antibodies for human therapy may diminish the chance of side effects due to an immunological reaction in a human individual against nonhuman sequences. In another embodiment, the antibody or antigen binding fragment thereof may be humanized. In another embodiment, the anti-RSV antibody or an antigen binding fragment thereof may be a chimeric antibody or antigen binding fragment thereof.

In some embodiments, the anti-RSV antibody or an antigen binding fragment thereof may have an IgG, IgA, IgM, or IgE isotype. In one embodiment, the anti-RSV antibody or an antigen binding fragment thereof has an IgG isotope. In some embodiments, the anti-RSV antibody or an antigen binding fragment thereof has a low or neutral isoelectric point (pI)—that is, the pH at which a protein has no net charge. In some embodiments, the pI of the anti-RSV antibody or an antigen binding fragment thereof is at pH 5.5, at least pH 6, at least pH 6.3, at least pH 6.4. In some embodiments, the pI of the anti-RSV antibody or an antigen binding fragment thereof is up to pH 6.7, up to pH 7, up to pH 7.5 In an exemplary embodiment, the anti-RSV antibody or an antigen binding fragment thereof may have a pI in the range of for example in the range about pH 5.5 to about pH 7.5. In an exemplary embodiment, the anti-RSV antibody or an antigen binding fragment thereof may have a pI in the range of pH 6.4 to pH 6.7. In one embodiment, the anti-RSV antibody or an antigen binding fragment thereof has a pI of pH 6.4.

In some embodiments, the anti-RSV antibody or an antigen binding fragment thereof includes nirsevimab (also known as MEDI8897) or an antigen binding fragment of nirsevimab. Nirsevimab is a recombinant human immunoglobulin (Ig) G1 kappa (κ monoclonal antibody derived from D25. Nirsevimab neutralizes RSV by binding to a highly conserved, neutralizing epitope on the prefusion conformation of the RSV F protein. This binding prevents the RSV F protein from mediating fusion between the viral and cellular membranes, which is an essential step for viral entry.

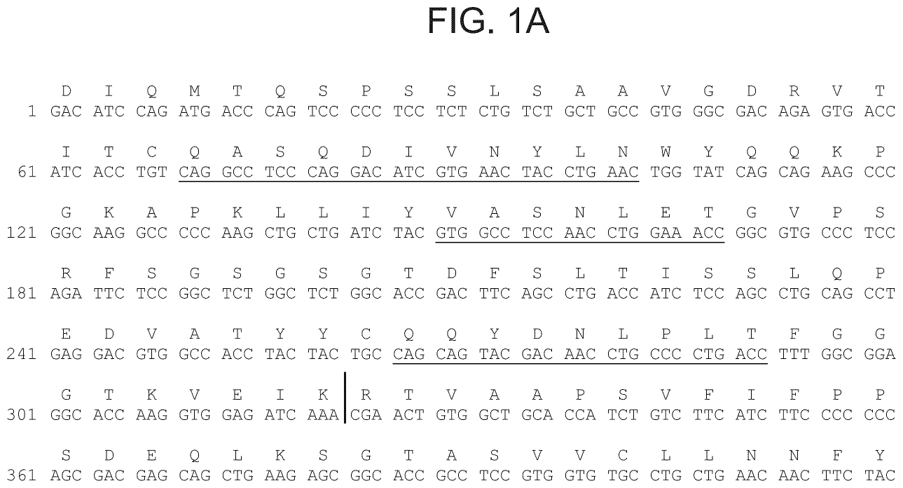

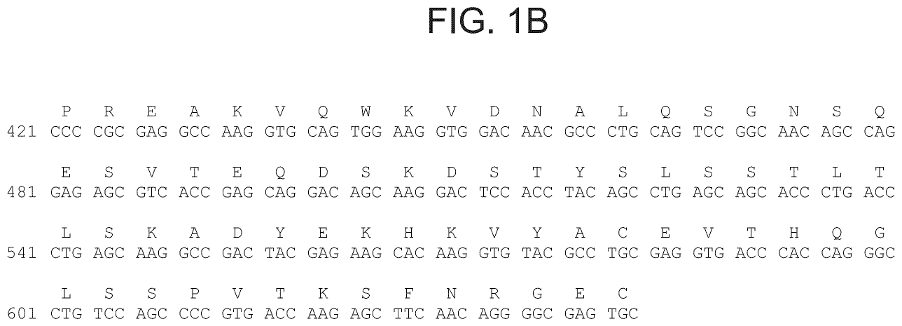

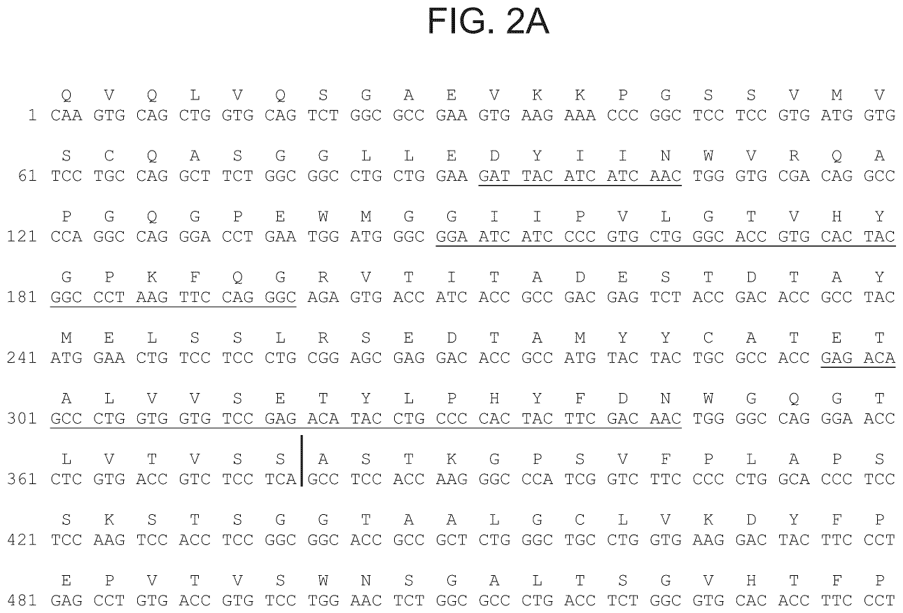

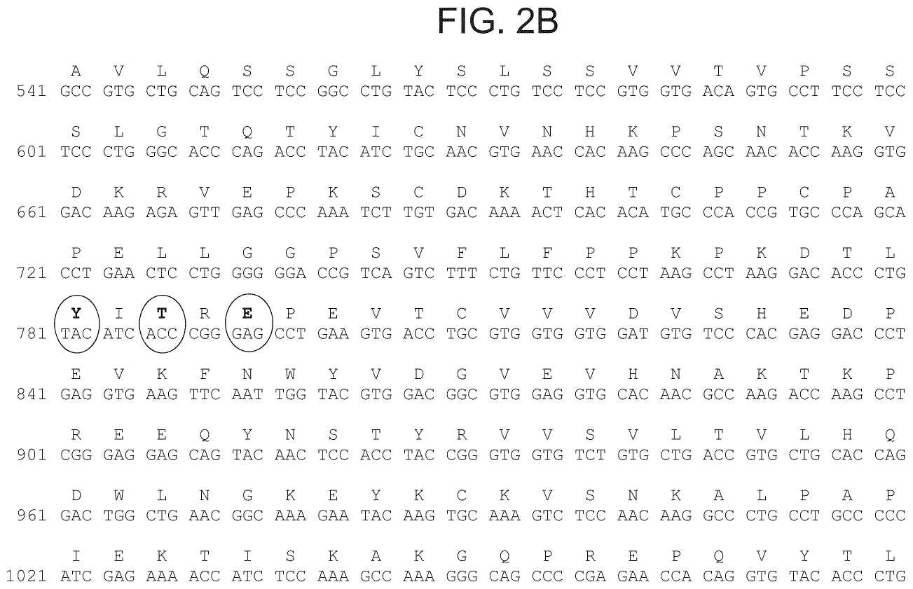

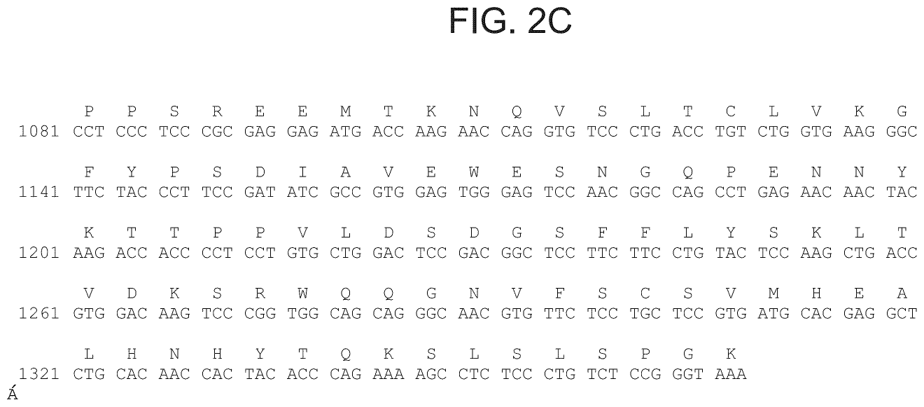

Nirsevimab has a full-length light chain amino acid sequence as shown in FIG. 1 (SEQ ID NO:1) and a full-length heavy chain amino acid sequence as shown in of FIG. 2 (SEQ ID NO:2).

Nirsevimab has the following CDR sequences: light chain CDR-L1 of QASQDIVNYLN (SEQ ID NO:3), light chain CDR-L2 of VASNLET (SEQ ID NO:4), light chain CDR-L3 of QQYDNLPLT (SEQ ID NO:5), heavy chain CDR-H1 of DYIIN (SEQ ID NO:6), heavy chain CDR-H2 of GIIPVLGTVHYGPKFQG (SEQ ID NO:7), and heavy chain CDR-H3 of ETALVVSETYLPHYFDN (SEQ ID NO:8). The 6 CDRS are underlined in FIG. 1 and FIG. 2.

Nirsevimab has a light chain variable sequence of amino acid residues 1 to 107 of FIG. 1 (SEQ ID NO:9) and a heavy chain variable sequence of amino acid residues 1 to 126 of FIG. 2 (SEQ ID NO:10).

Nucleotides encoding the amino acids of the full-length light chain of nirsevimab are also shown in in FIG. 1 (SEQ ID NO:11). Nucleotides encoding the amino acids of the full-length heavy chain of nirsevimab are also shown in in FIG. 2 (SEQ ID NO:12).

Nirsevimab is encoded by the RSV mAb 1G7 POE YTE vector, deposited with the American Type Culture Collection (ATCC®), 10801 University Boulevard, Manassas, Va. 20110-2209, USA under ATCC Patent Designation PTA-125141 on Sep. 21, 2018. This deposit is in accordance with the Budapest Treaty on the International Recognition of the Deposit of Microorganisms for the Purposes of Patent Procedure.

The nirsevimab-containing vector RSV mAb 1G7 pOE was deposited with the American Type Culture Collection (ATCC®), 10801 University Boulevard, Manassas, Va. 20110-2209, USA under ATCC Patent Designation PTA-125140 on Sep. 21, 2018. This deposit is in accordance with the Budapest Treaty on the International Recognition of the Deposit of Microorganisms for the Purposes of Patent Procedure.

Nirsevimab binds a highly conserved epitope on RSV F and neutralizes a diverse panel of RSV A and B strains with >50-fold higher activity than palivizumab. At similar serum concentrations, prophylactic administration of nirsevimab is 9-fold more potent than palivizumab at reducing pulmonary viral loads by >3 logs in cotton rats infected with either RSV A or B subtypes. (Zhu et al. Sci Transl Med. 2017; 9(388). pii:eaaj1928.) The nirsevimab antibody is engineered with 3 amino acid changes (M257Y/S259T/T261E [YTE]) in the highly conserved fragment crystallizable region. This YTE modification extends the serum half-life (t_{1/2}) of the antibody beyond the typical 21-28 days. In a phase 1, placebo-controlled study of healthy adults, nirsevimab had a favorable safety profile and an extended mean t_{1/2} of 85-117 days with increased levels of RSV-neutralizing antibodies detected in serum for more than 150 days. (Griffin et al. Antimicrob Agents Chemother. 2016; 61:e01714-e01716.)

In a phase 1b/2a dose-escalation study, healthy preterm infants with a gestational age of 32-35 weeks were randomized to receive a single intramuscular injection of nirsevimab (10 mg, 25 mg, or 50 mg) or placebo, and nirsevimab was observed to have a favorable safety profile in healthy preterm infants and to support protection from RSV for the duration of a typical 5-month season after a single 50 mg intramuscular (IM) dose. (Domachowske et al. Pediatr Infect Dis J. 2018; 37(9):886-892.)

Pharmacokinetic data in the phase 1b/2a study of nirsevimab demonstrated that a single 50 mg dose maintains antibody concentrations predictive of protection from RSV illness for at least 5 months in the majority of infants. (Domachowske et al. Pediatr Infect Dis J. 2018; 37(9):886-892.)

In some embodiments, the anti-RSV monoclonal antibody or the antigen binding fragment of the anti-RSV monoclonal antibody may include a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO:1 (the light chain sequence of nirsevimab) and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO:2 (the heavy chain chain sequence of nirsevimab). In some embodiments, the anti-RSV monoclonal antibody may include a sequence comprising SEQ ID NO:1 and/or SEQ ID NO:2.

In some embodiments, the anti-RSV monoclonal antibody or the antigen binding fragment of the anti-RSV monoclonal antibody may include a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence QASQDIVNYLN (SEQ ID NO:3); a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence VASNLET (SEQ ID NO:4); a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence QQYDNLPLT (SEQ ID NO:5); a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence DYIIN (SEQ ID NO:6), a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence GIIPVLGTVHYGPKFQG (SEQ ID NO:7); and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence ETALVVSETYLPHYFDN (SEQ ID NO:8).

In some embodiments, the anti-RSV monoclonal antibody or the antigen binding fragment of the anti-RSV monoclonal antibody may include QASQDIVNYLN (SEQ ID NO:3), VASNLET (SEQ ID NO:4), QQYDNLPLT (SEQ ID NO:5), DYIIN (SEQ ID NO:6), GIIPVLGTVHYGPKFQG (SEQ ID NO:7), and/or ETALVVSETYLPHYFDN (SEQ ID NO:8).

In some embodiments, the anti-RSV monoclonal antibody or the antigen binding fragment of the anti-RSV monoclonal antibody may include a light chain variable region CDR sequence or heavy chain variable region CDR sequence which differs by one amino acid from the corresponding CDR sequence of nirsevimab. In some embodiments, the anti-RSV monoclonal antibody or the antigen binding fragment of the anti-RSV monoclonal antibody may include a light chain variable region CDR sequence or heavy chain variable region CDR sequence which differs by two amino acids from the corresponding CDR sequence of nirsevimab.

In some embodiments, the anti-RSV monoclonal antibody or the antigen binding fragment of the anti-RSV monoclonal antibody may include a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO:9 and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO:10.

Unexpectedly, and as further described in the Examples of this disclosure, population pharmacokinetic and exposure-response analyses of all the accrued pharmacokinetic data (see Griffin et al. 2017, Antimicrob Agents Chemother. 61(3), pii: e01714-16; Domachowske et al. Pediatr Infect Dis J. 2018; 37(9):886-892; and Example 1) in healthy adults and infants and the efficacy data from the Phase 2b study in infants with various degrees of prematurity (see Example 1) revealed that while a 50 mg IM dose of nirsevimab maintained nirsevimab serum concentrations at a level expected to be protective throughout the RSV season, reduced efficacy was observed in heavier infants.

A target serum concentration of 6.8 μg/mL was selected for the Phase 2b Study based on RSV challenge studies in cotton rats, a model that has been proven to be a reliable predictor of target concentrations and was used for dose selection of palivizumab. A 50 mg IM dose was selected for the Phase 2b study based on the population-PK model used to identify a dose that would maintain nirsevimab serum concentrations above 6.8 μg/mL throughout a 5 month RSV season. And, indeed, Domachowske et al. reported that pharmacokinetic data in the phase 1b/2a study of nirsevimab demonstrated that a single 50 mg dose maintains antibody serum concentrations above 6.8 μg/mL for at least 5 months in the majority of infants. (Pediatr Infect Dis J. 2018; 37(9):886-892.)

However, data analysis and modeling completed using the data obtained in the Phase 2b study using the fixed 50-mg dose resulted in a newly defined clinically efficacious AUC_{0-∞} exposure target of 13.4 day·mg/mL. While a 50-mg dose resulted in achieving the efficacious AUC_{0-∞} exposure target of 13.4 day·mg/mL in 97% of infants weighing <5 kg, a 50 mg dose was suboptimal for >59% of infants weighing ≥5 kg.

Additional modeling (see Example 2) indicates that different fixed doses (including, for example, administered by weight) would ensure an adequate dose to maintain nirsevimab serum concentrations above the target AUC demonstrated to be clinically efficacious in the Phase 2b study throughout an RSV season. Those doses are: a single fixed 50 mg IM dose for infants up to 5 kg entering their first RSV season; a single fixed 100 mg IM dose for infants having a weight of at least 5 kg entering their first RSV season; and a single fixed 200 mg IM dose for infants entering their second RSV season. The body weight of majority of the infants in the second year of life at time of dosing is expected to be in a range of 10 kg to 15 kg.

### Methods of Administration

In one aspect, this disclosure describes methods of treating or preventing RSV infection in a patient in need thereof.

In some embodiments, the method includes determining the age of the patient. For example, it may be determined whether the patient is in the first year of life or in the second year of life. In some embodiments, the patient may have an age of at least 1 month, at least 3 months, at least 6 months, or at least 12 months (that is, one year). In some embodiments, the patient may have an age of up to one year, or up to two years.

In some embodiments, determining the age of the patient may include determining the gestational age of the patient. For example, in some embodiments, the patient may have a gestational age of at least 20 weeks, at least 21 weeks, at least 22 weeks, at least 23 weeks, at least 24 weeks, at least 25 weeks, at least 26 weeks, at least 27 weeks, at least 28 weeks, at least 29weeks, at least 30 weeks, at least 31 weeks, at least 32 weeks, at least 33 weeks, at least 34 weeks, at least 35 weeks, at least 36 weeks, at least 37 weeks, at least 38 weeks, or at least 39 weeks. In an exemplary embodiment, a patient may have a gestational age of at least 29 weeks.

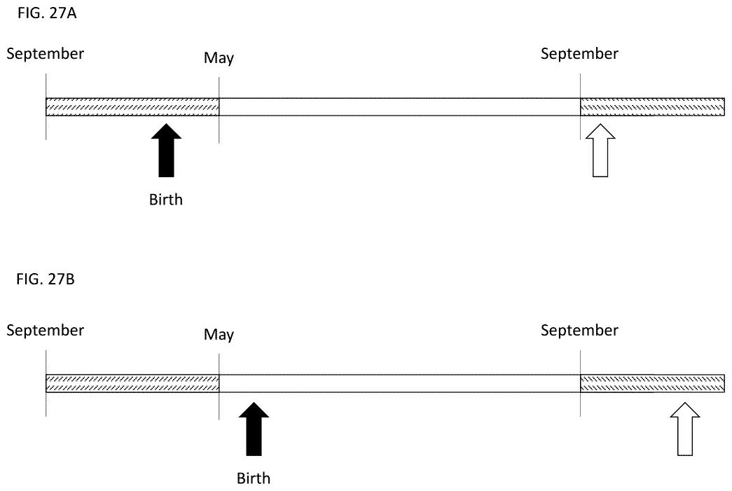

In some embodiments, the method includes determining whether the patient is experiencing the patient's first RSV season or second RSV season. For example, in the United States, where the RSV season onset ranges from mid-September to mid-November and season offset ranging from mid-April to mid-May, a patient born in April may be experiencing their second RSV season in October of the year of their birth (while ˜7 months old) (see FIG. 27A) while a patient born in June may be experiencing their first RSV in December of the same year (while also ˜7 months old) (see FIG. 27B).

In some embodiments, a patient experiencing the patient's first RSV season may have an age of at least 1 month, an age of at least 3 months, or an age of at least 6 months. In some embodiments, a patient experiencing the patient's second RSV season life has an age of at least 6 months and up to 2 years.

In some embodiments, the method includes determining the weight of the patient. For example, in some embodiments, the patient may have a weight of at least 4 kilograms (kg), at least 5 kg, at least 6 kg, at least 7 kg, at least 8 kg, at least 9 kg, or at least 10 kg. In some embodiments, the patient may have a weight of up to 5 kg, up to 6 kg, up to 7 kg, up to 8 kg, up to 9 kg, up to 10 kg, up to 11 kg, up to 12 kg, up to 13 kg, up to 14 kg, up to 15 kg, up to 16 kg, up to 17 kg, up to 18 kg, up to 19 kg, or up to 20 kg. In an exemplary embodiment, a patient may have a weight of at least 5 kg and up to 20 kg.

In some embodiments, the patient may have chronic lung disease (CLD). Premature babies are at increased risk for CLD due to the immaturity of their lung at birth and lung injury resulting from treatments such as use of a mechanical ventilator and/or use of a high concentration of oxygen. Infants with CLD are at particular risk of morbidity due to RSV infection. (Carpenter et al. 2004Pediatr Infect Dis J. 23(1 Suppl):S33-40.)

In some embodiments, the patient may have congenital heart disease (CHD). In some embodiments, the CHD may include hemodynamically significant CHD which may adversely affect pulmonary blood flow. Children with hemodynamically significant CHD have a greater rate of RSV-related hospitalization (Boyce et al. 2000 J Pediatr. 137(6):865-870), and children with CHD hospitalized for RSV are at increased risk of needing intensive care and mechanical ventilation (Altman et al. 2000 Pediatr Cardiol. 21(5):433-438).

In some embodiments, the patient may have Down's Syndrome. Children with Down's Syndrome have been reported to have a significantly higher risk of severe RSV infection than children without Down's Syndrome (Beckhaus et al., Pediatrics 2018; 142 (3):e20180225).

In some embodiments, the patient may exhibit an immunodeficiency. The immunodeficiency may be a primary immunodeficiency or an acquired immunodeficiency. Several studies have reported that RSV infections may be more frequent or more severe in infants with congenital or acquired immunodeficiencies (including, for example, infants with HIV infections or hematopoietic stem cell and solid organs transplant recipients) than in healthy infants (see, for example, Lanari et al., J Immunol Res. 2014; 2014:850831).

In some embodiments, the method will include administering a composition including a fixed dose of the anti-RSV antibody or an antigen binding fragment thereof including, for example, nirsevimab or an antigen binding fragment thereof. In some embodiments, a fixed dose of the anti-RSV antibody or an antigen binding fragment thereof may include a 50 mg dose, a 100 mg dose, a 150 mg dose, a 200 mg dose, a 250 mg dose, or a 300 mg dose.

In some embodiments, the method will include administering a composition depending on the patient's age and/or weight.

For example, in an exemplary embodiment, the method may include administering a composition including a fixed dose of 200 mg of nirsevimab in response to the patient being in the second year of life.

In another exemplary embodiment, the method may include administering a composition including a fixed dose of 200 mg of nirsevimab in response to the patient having a weight of at least 5 kg.

In an exemplary embodiment, the method may include administering a composition including a fixed dose of 50 mg or 100 mg of the anti-RSV antibody or an antigen binding fragment thereof in response to the patient being in the first year of life, depending on the weight of the patient. For example, in response to the patient having a weight of up to 5 kg, a composition including a fixed dose of 50 mg of nirsevimab may be administered. Additionally or alternatively in response to the patient having a weight of at least 5 kg, a composition including a fixed dose of 100 mg of nirsevimab may be administered.

In another exemplary embodiment, the method may include administering a composition including a fixed dose of 200 mg of the anti-RSV antibody or an antigen binding fragment thereof in response to the patient aged 3 months or older.

In a further exemplary embodiment, the method may include determining the age of the patient and administering a composition including a fixed dose of 200 mg of the anti-RSV antibody or an antigen binding fragment thereof in response to the patient aged 3 months or older, administering a composition including a fixed dose of 100 mg of the anti-RSV antibody or an antigen binding fragment thereof in response to the patient aged up to 3 months.

In an additional exemplary embodiment, the method may include determining the weight of the patient and administering a composition including a fixed dose of 200 mg of the anti-RSV antibody or an antigen binding fragment thereof in response to the patient having a weight of at least 5 kg, administering a composition including a fixed dose of 100 mg of the anti-RSV antibody or an antigen binding fragment thereof in response to the patient having a weight of up to 5 kg.

In some embodiments, the method will include administering a composition depending on the RSV season the patient is experiencing (that is, first RSV season or second RSV season) and/or the patient's weight.

For example, in an exemplary embodiment, the method may include administering a composition including a fixed dose of 100 mg of nirsevimab in response to the patient experiencing their first RSV season.

In another exemplary embodiment, the method may include administering a composition including a fixed dose of 200 mg of nirsevimab in response to the patient experiencing their second RSV season.

In an additional exemplary embodiment, the method may include determining the patient's RSV season and the weight of the patient. The method may further include administering a composition including a fixed dose of 100 mg of the anti-RSV antibody or an antigen binding fragment thereof in response to the patient experiencing their first RSV season and having a weight of at least 5 kg, and administering a composition including a fixed dose of 200 mg of the anti-RSV antibody or an antigen binding fragment thereof in response to the patient experiencing their second RSV season. In some embodiments, the method may further include administering a composition including a fixed dose of 50 mg of the anti-RSV antibody or an antigen binding fragment thereof in response to the patient experiencing their first RSV season and having a weight of up to 5 kg.

In some embodiments, the dose may be administered at the beginning of the RSV season. In the United States, for example, the RSV season onset ranges from mid-September to mid-November, with the season peak ranging from late December to mid-February, and season offset ranging from mid-April to mid-May. (Rose et al. 2018 MMWR Morb Mortal Wkly Rep. 67:71-76.) Lower latitudes, for example, Florida, have an earlier RSV season onset and longer duration than most regions of the country. (Id.) In contrast, in much of the southern hemisphere, RSV epidemics typically occur between May and September but in tropical or semitropical climates, RSV outbreaks are frequently associated with the rainy season. (Sricharoenchai et al. 2016 J Hum Virol Retrovirol 3(1): 00076.)

In some embodiments, the dose may be administered parenterally. In some embodiments, the dose may be administered intramuscularly.

In some embodiments, the patient exhibits AUC_{0-∞} of greater than 10 day·mg/mL, 11 day·mg/mL, 12 day·mg/mL, 13 day·mg/mL, or 14 day·mg/mL. In an exemplary embodiment, the patient exhibits AUC_{0-∞} of greater than 13.4 day·mg/mL. As further described in Example 2, a nirsevimab dose resulting in a serum AUC greater than 13.4 day·mg/mL throughout the typical 5-month RSV season is expected to provide optimal protection against RSV in infants during the first year of life and high-risk children during the second year of life.

In some embodiments, the method includes administering a composition including the anti-RSV antibody or an antigen binding fragment thereof prophylactically, to prevent or delay the development of RSV infection. Treatment that is prophylactic may be initiated before the patient manifests symptoms of infection with RSV. Administration may be performed before, during, or after the diagnosis or development of symptoms of infection with RSV. Treatment initiated after the development of symptoms may result in decreasing the severity of the symptoms of RSV, or completely removing the symptoms of RSV.

Administration of a composition including the anti-RSV antibody or an antigen binding fragment thereof can occur before, during, and/or after other treatments. Such combination therapy can involve the administration of the anti-RSV antibody or an antigen binding fragment thereof during and/or after the use of another anti-viral compound. The administration of the anti-RSV antibody or an antigen binding fragment thereof can be separated in time from the administration of other agents by hours, days, or even weeks.

### Compositions

In another aspect, the disclosure describes a composition including the anti-RSV antibody or an antigen binding fragment thereof. In some embodiments, the composition includes nirsevimab or an antigen binding fragment thereof. In some embodiments, the composition includes a therapeutically effective amount of the anti-RSV antibody or an antigen binding fragment thereof. In some embodiments, the composition may include a formulation as described in WO 2018/160722 A1. In some embodiments, the composition includes a pharmaceutical composition. A pharmaceutical composition may further include a pharmaceutically acceptable diluent, carrier, solubilizer, emulsifier, preservative, and/or adjuvant. In addition, the disclosure provides methods of treating a patient by administering such pharmaceutical composition.

In certain embodiments, acceptable materials included in the composition are nontoxic to recipients at the dosages and concentrations employed. In certain embodiments, the pharmaceutical composition may contain formulation materials for modifying, maintaining or preserving, for example, the pH, osmolality, viscosity, clarity, color, isotonicity, odor, sterility, stability, rate of dissolution, or release, adsorption, or penetration of the composition. In such embodiments, suitable formulation materials include, but are not limited to, amino acids (such as glycine, glutamine, asparagine, arginine, or lysine); antimicrobials; antioxidants (such as ascorbic acid, sodium sulfite, or sodium hydrogen-sulfite); buffers (such as borate, bicarbonate, Tris-HCl, citrates, phosphates, or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin, or hydroxypropyl-beta-cyclodextrin); fillers; monosaccharides; disaccharides; and other carbohydrates (such as glucose, sucrose, mannose, or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate-20, polysorbate-80, triton, tromethamine, lecithin, cholesterol, tyloxapal); stability enhancing agents (such as sucrose or sorbitol); tonicity enhancing agents (such as alkali metal halides, sodium or potassium chloride, mannitol sorbitol); delivery vehicles; diluents; excipients and/or pharmaceutical adjuvants.

A suitable vehicle or carrier may be water for injection, physiological saline solution or artificial cerebrospinal fluid. In some embodiments the vehicle or carrier may be supplemented with other materials common in compositions for parenteral administration. Neutral buffered saline or saline mixed with serum albumin are further exemplary vehicles.

In some embodiments, the composition includes a buffer. Exemplary buffers include Tris buffer of pH 7.0-8.5, acetate buffer of pH 4.0-5.5, or histidine buffer of pH 5.5-7.4. In some embodiments, the composition includes histidine or histidine hydrochloride or a mixture thereof. When the buffer includes an amino acid (e.g., histidine), the amino acid or amino acid salt may include the physiological active (e.g., L-form) of the amino acid. In some embodiments, a buffer may be included at a concentration of at least 20 mM, at least 30 mM, at least 40 mM, at least 50 mM, at least 60 mM, at least 70 mM, at least 80 mM, at least 90 mM, at least 100 mM, at least 110 mM, at least 120 mM, at least 130 mM, at least 140 mM, or at least 150 mM. In some embodiments, a buffer may be included at a concentration of up to 50 mM, up to 60 mM, up to 70 mM, up to 80 mM, up to 90 mM, up to 100 mM, up to 110 mM, up to 120 mM, up to 130 mM, up to 140 mM, up to 150 mM, or up to 160 mM. In an exemplary embodiment, the buffer includes 30 mM L-histidine/L-histidine hydrochloride.

In some embodiments, the composition may include a humectant including, for example, sorbitol or a suitable substitute therefor.

The formulation components may be present in concentrations that are acceptable to the site of administration. In certain embodiments, a buffer may be used to maintain the composition at physiological pH or at a slightly lower that physiological pH. In some embodiments, the pH of the composition may be at least 5, at least 5.1, at least 5.2, at least 5.3, at least 5.4, at least 5.5, at least 5.6, at least 5.7, at least 5.8, at least 5.9, at least 6.0, at least 6.1, at least 6.2, at least 6.3, at least 6.4, at least 6.5, at least 6.6, at least 6.7, at least 6.8, at least 6.9, at least 7.0, at least 7.1, at least 7.2, at least 7.3, at least 7.4, at least 7.5, at least 7.6, at least 7.7, at least 7.8, or at least 7.9. In some embodiments, the pH of the composition may be up to 5.1, up to 5.2, up to 5.3, up to 5.4, up to 5.5, up to 5.6, up to 5.7, up to 5.8, up to 5.9, up to 6.0, up to 6.1, up to 6.2, up to 6.3, up to 6.4, up to 6.5, up to 6.6, up to 6.7, up to 6.8, up to 6.9, up to 7.0, up to 7.1, up to 7.2, up to 7.3, up to 7.4, up to 7.5, up to 7.6, up to 7.7, up to 7.8, up to 7.9, or up to 8.0. In an exemplary embodiment, the pH of the composition may be in a range of from 5 to 8. In an exemplary embodiment, the pH of the composition may be in a range of from 5.5 to 6.5. In an exemplary embodiment, the pH of the composition may be 6.0.

In some embodiments, the composition may include an ionic excipient. An ionic excipient may be included in an antibody formulation for the purpose of changing the charge state of the antibody in the formulation, for changing the distribution of the antibody in the formulation, and/or for colloidally stabilizing the antibody in the formulation. In some embodiments, the ionic excipient may include a charged amino acid including, for example, lysine and/or arginine. In some embodiments, the ionic excipient may include a salt including, for example arginine hydrochloride (arginine-HCl), lysine hydrochloride (lysine-HCl), or sodium chloride (NaCl). In some embodiments, the amino acid or amino acid salt may include the physiological active (e.g., L-form) of the amino acid. In some embodiments, an ionic excipient may be included at a concentration of at least 20 mM, at least 30 mM, at least 40 mM, at least 50 mM, at least 60 mM, at least 70 mM, at least 80 mM, at least 90 mM, at least 100 mM, at least 110 mM, at least 120 mM, at least 130 mM, at least 140 mM, or at least 150 mM. In some embodiments, an ionic excipient may be included at a concentration of up to 50 mM, up to 60 mM, up to 70 mM, up to 80 mM, up to 90 mM, up to 100 mM, up to 110 mM, up to 120 mM, up to 130 mM, up to 140 mM, up to 150 mM, or up to 160 mM. In an exemplary embodiment, an ionic excipient may be present at a concentration in a range of 50 mM to 150 mM. In an exemplary embodiment, an ionic excipient may be present at a concentration in a range of 75 mM to 100 mM. In exemplary embodiments, the ionic excipient may include L-arginine hydrochloride present at a concentration of 75 mM or 80 mM.

In some embodiments, a composition that includes the anti-RSV antibody or an antigen binding fragment thereof may further include a sugar including, for example, sucrose. In some embodiments, the composition may include up to 0.5% (w/v) sucrose, up to 1% (w/v) sucrose, up to 5% (w/v) sucrose, up to 10% (w/v) sucrose, or up to 15% (w/v) sucrose. In some embodiments, a sugar may be included at a concentration of at least 50 mM, at least 60 mM, at least 70 mM, at least 80 mM, at least 90 mM, at least 100 mM, at least 110 mM, at least 120 mM, at least 130 mM, at least 140 mM, or at least 150 mM. In some embodiments, a sugar may be included at a concentration of up to 60 mM, up to 70 mM, up to 80 mM, up to 90 mM, up to 100 mM, up to 110 mM, up to 120 mM, up to 130 mM, up to 140 mM, up to 150 mM, or up to 160 mM. In an exemplary embodiment, the sugar includes sucrose at a concentration in a range of 100 mM to 140 mM. For example, the composition may include sucrose at a concentration of 120 mM.

In some embodiments, a composition that includes the anti-RSV antibody or an antigen binding fragment thereof may further include a surfactant including, for example, a polysorbate. A polysorbate may include, for example, polysorbate-20, polysorbate-40, polysorbate-60, and polysorbate-80. In some embodiments, the surfactant may be included at a concentration of 0.0001% (w/v), at least 0.001% (w/v), at least 0.002% (w/v), at least 0.01% (w/v), at least 0.02% (w/v), at least 0.03% (w/v), at least 0.04% (w/v), at least 0.05% (w/v), at least 0.06% (w/v), at least 0.07% (w/v), at least 0.08% (w/v), at least 0.09% (w/v), or at least 0.1% (w/v). In some embodiments, the surfactant may be included at a concentration of up to 0.0001% (w/v), up to 0.0005% (w/v), up to 0.001% (w/v), up to 0.002% (w/v), up to 0.01% (w/v), up to 0.02% (w/v), up to 0.03% (w/v), up to 0.04% (w/v), up to 0.05% (w/v), up to 0.06% (w/v), up to 0.07% (w/v), up to 0.08% (w/v), up to 0.09% (w/v), or up to 0.1% (w/v). For example, in an exemplary embodiment, a surfactant may be included in a concentration in a range of 0.001% (w/v) to 0.5% (w/v), in a range of 0.002% (w/v) to 0.1% (w/v), or in a range of 0.01% (w/v) to 0.05% (w/v). In an exemplary embodiment, polysorbate-80 is included in a range of 0.01% (w/v) to 0.05% (w/v). In a further exemplary embodiment, 0.02% (w/v) polysorbate-80 is included in the composition. In another exemplary embodiment, 0.04% (w/v) polysorbate-80 is included in the composition.

In some embodiments, the anti-RSV antibody or an antigen binding fragment thereof may be at a concentration of at least 50 mg/mL, at least 60 mg/mL, at least 70 mg/mL, at least 80 mg/mL, at least 90 mg/mL, at least 100 mg/mL, at least 110 mg/mL, at least 120 mg/mL, at least 130 mg/mL, at least 140 mg/mL, or at least 150 mg/mL. In some embodiments, the anti-RSV antibody or an antigen binding fragment thereof may be at a concentration of up to 60 mg/mL, up to 70 mg/mL, up to 80 mg/mL, up to 90 mg/mL, up to 100 mg/mL, up to 110 mg/mL, up to 120 mg/mL, up to 130 mg/mL, up to 140 mg/mL, up to 150 mg/mL, or up to 160 mg/mL. In an exemplary embodiment, the anti-RSV antibody or an antigen binding fragment thereof may present at a concentration in a range of 100 mg/ml to 165 mg/ml. In an exemplary embodiment, nirsevimab may be at a concentration of 100 mg/mL.

In some embodiments, the composition may be stored at −20° C. to −70° C.

In some embodiments, the composition may be stored at 2° C. to 8° C. In some embodiments, the formulations described herein are stable for extended periods of storage at room temperature or at a temperature in a range of 2° C. to 8° C., including, for example, 5° C. As used herein, room temperature is generally a temperature in the range of 22° C. to 25° C. Suitably the pharmaceutical formulations are stable after storage at a temperature in a range of 2° C. to 8° C. (including, for example, 5° C.) for at least one month, at least three months, or at least six months. As used herein, the term “stable” for a period of storage (or “stability”) is used to indicate that the formulations resist aggregation, degradation, half antibody formation, and/or fragmentation. The stability of the monoclonal antibodies can be assessed by degrees of aggregation, degradation, half antibody formation or fragmentation, as measured by high performance size exclusion chromatography (HPSEC), static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference.

When parenteral administration is contemplated, the therapeutic compositions for use may be provided in the form of a pyrogen-free, parenterally acceptable aqueous solution including the anti-RSV antibody or an antigen binding fragment thereof in a pharmaceutically acceptable vehicle. In some embodiments, a particularly suitable vehicle for parenteral injection is sterile distilled water in which the antibody is formulated as a sterile, isotonic solution, properly preserved. Additionally or alternatively, formulations suitable for parenteral administration may include a sterile aqueous preparation of the anti-RSV antibody or an antigen binding fragment thereof, or dispersions of sterile powders of the anti-RSV antibody or an antigen binding fragment thereof, which are may be isotonic with the blood of the recipient. Isotonic agents that can be included in the liquid preparation include sugars, buffers, and sodium chloride. Solutions of the anti-RSV antibody or an antigen binding fragment thereof can be prepared in water, optionally mixed with a nontoxic surfactant. Dispersions of the anti-RSV antibody or an antigen binding fragment thereof can be prepared in water, ethanol, a polyol (such as glycerol, propylene glycol, liquid polyethylene glycols, and the like), vegetable oils, glycerol esters, and mixtures thereof. The ultimate dosage form may, in some embodiments, be sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the anti-RSV antibody or an antigen binding fragment thereof, for example, by filter sterilization. Methods for preparing powders include vacuum drying and freeze drying of the sterile injectable solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the anti-RSV antibody or an antigen binding fragment thereof over a prolonged period can be achieved by including agents for delaying, for example, aluminum monostearate and gelatin.

In certain embodiments, the composition can include a formulation of the anti-RSV antibody or an antigen binding fragment thereof with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the antibody.

In some embodiments, the composition may be conveniently presented in unit dosage form. For example, an exemplary pharmaceutical unit dose includes a 50 mg dose of nirsevimab; a 100 mg dose of nirsevimab, or a 200 mg dose of nirsevimab. Such a unit dose can be prepared by any of the methods well-known in the art of pharmacy. In some embodiments, the unit dose is suitable for parenteral administration including, for example, intramuscular administration

### Exemplary Method Embodiments—Patient Age and Weight

1. A method of treating or preventing RSV infection in a patient in need thereof, the method comprising:
   determining the age of the patient;
   in response to the patient being in the second year of life, administering a composition comprising a fixed dose of 200 milligrams (mg) of an anti-RSV monoclonal antibody or an antigen binding fragment thereof;
   in response to the patient being in the first year of life, determining the patient's weight; and
   in response to the patient having a weight of at least 5 kilograms (kg), administering a composition comprising a fixed dose of 100 mg of an anti-RSV monoclonal antibody or an antigen binding fragment thereof,
   wherein the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320, or both.
2. The method of Embodiment 1, wherein the anti-RSV monoclonal antibody or the antigen binding fragment of the anti-RSV monoclonal antibody comprises a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO:1and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO:2.
3. The method of any one of the preceding Embodiments, wherein the anti-RSV monoclonal antibody or the antigen binding fragment of the anti-RSV monoclonal antibody comprises a sequence comprising SEQ ID NO:1 and/or SEQ ID NO:2.
4. The method of any one of the preceding Embodiments, wherein the anti-RSV monoclonal antibody or the antigen binding fragment of the anti-RSV monoclonal antibody comprises:
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence QASQDIVNYLN (SEQ ID NO:3);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence VASNLET (SEQ ID NO:4);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence QQYDNLPLT (SEQ ID NO:5);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence DYIIN (SEQ ID NO:6);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence GIIPVLGTVHYGPKFQG (SEQ ID NO:7); and/or
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence ETALVVSETYLPHYFDN (SEQ ID NO:8).
5. The method of any one of the preceding Embodiments, wherein the anti-RSV monoclonal antibody or the antigen binding fragment of the anti-RSV monoclonal antibody comprises QASQDIVNYLN (SEQ ID NO:3), VASNLET (SEQ ID NO:4), QQYDNLPLT (SEQ ID NO:5), DYIIN (SEQ ID NO:6), GIIPVLGTVHYGPKFQG (SEQ ID NO:7), and/or ETALVVSETYLPHYFDN (SEQ ID NO:8).
6. The method of any one of the preceding Embodiments, wherein the anti-RSV monoclonal antibody or the antigen binding fragment of the anti-RSV monoclonal antibody comprises QASQDIVNYLN (SEQ ID NO:3), VASNLET (SEQ ID NO:4), QQYDNLPLT (SEQ ID NO:5), DYIIN (SEQ ID NO:6), GIIPVLGTVHYGPKFQG (SEQ ID NO:7), and ETALVVSETYLPHYFDN (SEQ ID NO:8).
7. The method of any one of the preceding Embodiments, wherein the anti-RSV monoclonal antibody comprises a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO:9 and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO:10.
8. The method of any one of the preceding Embodiments, wherein the anti-RSV monoclonal antibody comprises nirsevimab or wherein the antigen binding fragment of the anti-RSV monoclonal antibody comprises an antigen binding fragment of nirsevimab.
9. The method of any one of the preceding Embodiments, wherein the patient having a weight of at least 5 kg has a weight of up to 10 kg, up to 15 kg, or up to 20 kg.
10. The method of any one of the preceding Embodiments, wherein the patient being in the first year of life has an age of at least 1 month, an age of at least 3 months, or an age of at least 6 months.
11. The method of any one of the preceding Embodiments, wherein the patient in the second year of life has an age of at least one year and up to 2 years.
12. The method of any one of the preceding Embodiments, wherein the patient has a gestational age of at least 29 weeks.
13. The method of any one of the preceding Embodiments, wherein the patient exhibits AUC_{0-∞} of greater than 13.4 day·mg/mL.
14. The method of any one of the preceding Embodiments, the method comprising administering the dose at the beginning of the RSV season.
15. The method of any one of the preceding Embodiments, the method comprising administering the dose intramuscularly.
16. The method of any one of the preceding Embodiments, wherein the patient exhibits Down's Syndrome, an immunodeficiency, congenital lung disease, or congenital heart disease or a combination thereof.
17. The method of any one of the preceding Embodiments, wherein the patient exhibits congenital lung disease or congenital heart disease or both.
18. The method of any one of the preceding Embodiments, the method comprising administering the anti-RSV monoclonal antibody or an antigen binding fragment thereof in a composition comprising at least one of an ionic excipient, a buffer, a sugar, and a surfactant.
19. The method of Embodiment 28, wherein the ionic excipient comprises L-arginine hydrochloride at a concentration of 80 mM.
20. The pharmaceutical unit dose of Embodiment 18 or Embodiment 19, wherein the buffer comprises 30 mM L-histidine/L-histidine hydrochloride, the sugar comprises 120 mM sucrose, and the surfactant comprises polysorbate-80 in a range of 0.01% (w/v) to 0.05% (w/v).
21. The method of any one of Embodiments 18 to 20, wherein the anti-RSV monoclonal antibody or an antigen binding fragment thereof in the composition is stable at 2° C. to 8° C. for at least 3 months as determined by high performance size exclusion chromatography (HPSEC).
22. The method of any one of Embodiments 18 to 21, wherein the composition has a pH in a range of from 5.5 to 6.5.
23. The method of any one of Embodiments 18 to 22, wherein the the anti-RSV monoclonal antibody or an antigen binding fragment thereof is present in the composition at a concentration of 100 mg/mL.

### Exemplary Method Embodiments—Patient Age or Weight

1. A method of treating or preventing RSV infection in a patient in need thereof, the method comprising administering to a patient having a weight of at least 5 kilograms (kg) a fixed dose of 200 milligrams (mg) of an anti-RSV monoclonal antibody or an antigen binding fragment thereof, wherein the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320, or both.
2. A method of treating or preventing RSV infection in a patient in need thereof, the method comprising:
   determining the weight of a patient;
   administering to a patient having a weight of at least 5 kilograms (kg) a fixed dose of 200 milligrams (mg) of an anti-RSV monoclonal antibody or an antigen binding fragment thereof, wherein the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320, or both;
   administering to a patient having a weight of up to 5 kg a fixed dose of 100 mg of an anti-RSV monoclonal antibody or an antigen binding fragment thereof, wherein the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320, or both.
3. The method of either of the preceding Embodiments, wherein the patient having a weight of at least 5 kg has a weight of up to 10 kg, up to 15 kg, or up to 20 kg.
4. A method of treating or preventing RSV infection in a patient in need thereof, the method comprising
   determining the age of the patient; and
   administering a fixed dose of 200 milligrams (mg) of an anti-RSV monoclonal antibody or an antigen binding fragment thereof to a patient aged 3 months or older, wherein the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320 or both.
5. The method of Embodiment 4, where the patient is 6 months or older.
6. The method of Embodiment 4 or Embodiment 5, wherein the patient is up to two years old.
7. A method of treating or preventing RSV infection in a patient in need thereof, the method comprising:
   determining the age of a patient;
   administering a fixed dose of 200 milligrams (mg) of an anti-RSV monoclonal antibody or an antigen binding fragment thereof to a patient aged 3 months or older;
   administering a fixed dose of 100 mg of an anti-RSV monoclonal antibody or an antigen binding fragment thereof to a patient aged up to 3 months,
   wherein the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320, or both.
8. The method of Embodiment 7, wherein the patient aged 3 months or older is up to two years old.
9. A method of treating or preventing RSV infection in a patient in need thereof, the method comprising:
   determining the age of a patient;
   administering a fixed dose of 200 milligrams (mg) of an anti-RSV monoclonal antibody or an antigen binding fragment thereof to a patient aged 6 months or older;
   administering a fixed dose of 100 mg of an anti-RSV monoclonal antibody or an antigen binding fragment thereof to a patient aged up to 6 months,
   wherein the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320, or both.
10. The method of Embodiment 9, wherein the patient aged 6 months or older is up to two years old
11. The method of any one of the preceding Embodiments, wherein the patient has a gestational age of at least 29 weeks.
12. The method of any one of the preceding Embodiments, wherein the patient exhibits AUC_{0-∞} of greater than 13.4 day·mg/mL.
13. The method of any one of the preceding Embodiments, the method comprising administering the dose at the beginning of the RSV season.
14. The method of any one of the preceding Embodiments, the method comprising administering the dose intramuscularly.
15. The method of any one of the preceding Embodiments, wherein the patient exhibits Down's Syndrome, an immunodeficiency, congenital lung disease, or congenital heart disease or a combination thereof.
16. The method of any one of the preceding Embodiments, wherein the patient exhibits congenital lung disease or congenital heart disease or both.
17. The method of any one of the preceding Embodiments, the method comprising administering the anti-RSV monoclonal antibody or an antigen binding fragment thereof in a composition comprising at least one of an ionic excipient, a buffer, a sugar, and a surfactant.
18. The method of Embodiment 17, wherein the ionic excipient comprises L-arginine hydrochloride at a concentration of 80 mM.
19. The method of Embodiment 17 or Embodiment 18, wherein the buffer comprises 30 mM L-histidine/L-histidine hydrochloride, the sugar comprises 120 mM sucrose, and/or the surfactant comprises polysorbate 80 in a range of 0.01% (w/v) to 0.05% (w/v).
20. The method of any one of Embodiments 18 to 19, wherein the anti-RSV monoclonal antibody or an antigen binding fragment thereof in the composition is stable at 2° C. to 8° C. for at least 3 months as determined by high performance size exclusion chromatography (HPSEC).
21. The method of any one of Embodiments 18 to 20, wherein the composition has a pH in a range of from 5.5 to 6.5.
22. The method of any one of Embodiments 18 to 21, wherein the the anti-RSV monoclonal antibody or an antigen binding fragment thereof is present in the composition at a concentration of 100 mg/mL.
23. The method of any one of the preceding Embodiments, wherein the anti-RSV monoclonal antibody or the antigen binding fragment of the anti-RSV monoclonal antibody comprises a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 1 and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical SEQ ID NO:2.
24. The method of any one of the preceding Embodiments, wherein the anti-RSV monoclonal antibody or the antigen binding fragment of the anti-RSV monoclonal antibody comprises a sequence comprising SEQ ID NO:1 and/or SEQ ID NO:2.
25. The method of any one of the preceding Embodiments, wherein the anti-RSV monoclonal antibody or the antigen binding fragment of the anti-RSV monoclonal antibody comprises:
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence QASQDIVNYLN (SEQ ID NO:3);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence VASNLET (SEQ ID NO:4);
a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence QQYDNLPLT (SEQ ID NO:5);
a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence DYIIN (SEQ ID NO:6);
a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence GIIPVLGTVHYGPKFQG (SEQ ID NO:7); and/or
a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence ETALVVSETYLPHYFDN (SEQ ID NO:8).
26. The method of any one of the preceding Embodiments, wherein the anti-RSV monoclonal antibody or the antigen binding fragment of the anti-RSV monoclonal antibody comprises QASQDIVNYLN (SEQ ID NO:3), VASNLET (SEQ ID NO:4), QQYDNLPLT (SEQ ID NO:5), DYIIN (SEQ ID NO:6), GIIPVLGTVHYGPKFQG (SEQ ID NO:7), and/or ETALVVSETYLPHYFDN (SEQ ID NO:8).
27. The method of any one of the preceding Embodiments, wherein the anti-RSV monoclonal antibody or the antigen binding fragment of the anti-RSV monoclonal antibody comprises QASQDIVNYLN (SEQ ID NO:3), VASNLET (SEQ ID NO:4), QQYDNLPLT (SEQ ID NO:5), DYIIN (SEQ ID NO:6), GIIPVLGTVHYGPKFQG (SEQ ID NO:7), and ETALVVSETYLPHYFDN (SEQ ID NO:8).
28. The method of any one of the preceding Embodiments, wherein the anti-RSV monoclonal antibody comprises a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO:9 and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO:10.
29. The method of any one of the preceding Embodiments, wherein the anti-RSV monoclonal antibody comprises nirsevimab or wherein the antigen binding fragment of the anti-RSV monoclonal antibody comprises an antigen binding fragment of nirsevimab.

### Exemplary Method Embodiments—Patient's RSV Season and Weight

1. A method of treating or preventing RSV infection in a patient in need thereof, the method comprising:
   determining whether the patient is experiencing their first or second RSV season;
   in response to the patient experiencing their second RSV season, administering a composition comprising a fixed dose of 200 milligrams (mg) of an anti-RSV monoclonal antibody or an antigen binding fragment thereof;
   in response to the patient experiencing their first RSV season, determining the patient's weight; and
   in response to the patient having a weight of at least 5 kilograms (kg), administering a composition comprising a fixed dose of 100 mg of an anti-RSV monoclonal antibody or an antigen binding fragment thereof,
   wherein the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320, or both.
2. The method of Embodiment 1, wherein the anti-RSV monoclonal antibody or the antigen binding fragment of the anti-RSV monoclonal antibody comprises a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO:1 and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO:2.
3. The method of any one of the preceding Embodiments, wherein the anti-RSV monoclonal antibody or the antigen binding fragment of the anti-RSV monoclonal antibody comprises a sequence comprising SEQ ID NO:1 and/or SEQ ID NO:2.
4. The method of any one of the preceding Embodiments, wherein the anti-RSV monoclonal antibody or the antigen binding fragment of the anti-RSV monoclonal antibody comprises:
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence QASQDIVNYLN (SEQ ID NO:3);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence VASNLET (SEQ ID NO:4);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence QQYDNLPLT (SEQ ID NO:5);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence DYIIN (SEQ ID NO:6);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence GIIPVLGTVHYGPKFQG (SEQ ID NO:7); and/or
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence ETALVVSETYLPHYFDN (SEQ ID NO:8).
5. The method of any one of the preceding Embodiments, wherein the anti-RSV monoclonal antibody or the antigen binding fragment of the anti-RSV monoclonal antibody comprises QASQDIVNYLN (SEQ ID NO:3), VASNLET (SEQ ID NO:4), QQYDNLPLT (SEQ ID NO:5), DYIIN (SEQ ID NO:6), GIIPVLGTVHYGPKFQG (SEQ ID NO:7), and/or ETALVVSETYLPHYFDN (SEQ ID NO:8).
6. The method of any one of the preceding Embodiments, wherein the anti-RSV monoclonal antibody or the antigen binding fragment of the anti-RSV monoclonal antibody comprises QASQDIVNYLN (SEQ ID NO:3), VASNLET (SEQ ID NO:4), QQYDNLPLT (SEQ ID NO:5), DYIIN (SEQ ID NO:6), GIIPVLGTVHYGPKFQG (SEQ ID NO:7), and ETALVVSETYLPHYFDN (SEQ ID NO:8).
7. The method of any one of the preceding Embodiments, wherein the anti-RSV monoclonal antibody comprises a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO:9 and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO:10.
8. The method of any one of the preceding Embodiments, wherein the anti-RSV monoclonal antibody comprises nirsevimab or wherein the antigen binding fragment of the anti-RSV monoclonal antibody comprises an antigen binding fragment of nirsevimab.
9. The method of any one of the preceding Embodiments, wherein the patient having a weight of at least 5 kg has a weight of up to 10 kg, up to 15 kg, or up to 20 kg.
10. The method of any one of the preceding Embodiments, wherein the patient experiencing their first RSV season has an age of at least 1 month, an age of at least 3 months, or an age of at least 6 months.
11. The method of any one of the preceding Embodiments, wherein the patient experiencing their second RSV season life has an age of at least 6 months and up to 2 years.
12. The method of any one of the preceding Embodiments, wherein the patient has a gestational age of at least 29 weeks.
13. The method of any one of the preceding Embodiments, wherein the patient exhibits AUC_{0-∞} of greater than 13.4 day·mg/mL.
14. The method of any one of the preceding Embodiments, the method comprising administering the dose at the beginning of the RSV season.
15. The method of any one of the preceding Embodiments, the method comprising administering the dose intramuscularly.
16. The method of any one of the preceding Embodiments, wherein the patient exhibits Down's Syndrome, immunodeficiency, congenital lung disease, or congenital heart disease or a combination thereof.
17. The method of any one of the preceding Embodiments, wherein the patient exhibits congenital lung disease or congenital heart disease or both.
18. The method of any one of the preceding Embodiments, the method comprising administering the anti-RSV monoclonal antibody or an antigen binding fragment thereof in a composition comprising at least one of an ionic excipient, a buffer, a sugar, and a surfactant.
19. The method of Embodiment 28, wherein the ionic excipient comprises L-arginine hydrochloride at a concentration of 80 mM.
20. The pharmaceutical unit dose of Embodiment 18 or Embodiment 19, wherein the buffer comprises 30 mM L-histidine/L-histidine hydrochloride, the sugar comprises 120 mM sucrose, and the surfactant comprises polysorbate-80 in a range of 0.01% (w/v) to 0.05% (w/v).
21. The method of any one of Embodiments 18 to 20, wherein the anti-RSV monoclonal antibody or an antigen binding fragment thereof in the composition is stable at 2° C. to 8° C. for at least 3 months as determined by high performance size exclusion chromatography (HPSEC).
22. The method of any one of Embodiments 18 to 21, wherein the composition has a pH in a range of from 5.5 to 6.5.
23. The method of any one of Embodiments 18 to 22, wherein the the anti-RSV monoclonal antibody or an antigen binding fragment thereof is present in the composition at a concentration of 100 mg/mL.

### Exemplary Method Embodiments—Patient's RSV Season

1. A method of treating or preventing RSV infection in a patient in need thereof, the method comprising
   determining whether the patient is experiencing their first or second RSV season; and
   administering a fixed dose of 200 milligrams (mg) of an anti-RSV monoclonal antibody or an antigen binding fragment thereof to a patient experiencing their second RSV season, wherein the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320 or both.
2. The method of Embodiment 1, where the patient is 3 months or older.
3. The method of Embodiment 1, where the patient is 6 months or older.
4. The method of any one of Embodiments 1 to 3, wherein the patient is up to two years old.
5. A method of treating or preventing RSV infection in a patient in need thereof, the method comprising:
   determining whether the patient is experiencing their first or second RSV season;
   administering a fixed dose of 200 milligrams (mg) of an anti-RSV monoclonal antibody or an antigen binding fragment thereof to a patient, wherein the patient is experiencing their second RSV season;
   administering a fixed dose of 100 mg of an anti-RSV monoclonal antibody or an antigen binding fragment thereof to a patient, wherein the patient is experiencing their first RSV season;
   wherein the anti-RSV monoclonal antibody or an antigen binding fragment thereof has an IC50 of 5.0 ng/ml or less in a neutralization assay of RSV A, or has an IC50 of 3.0 ng/ml or less in a neutralization assay of RSV B9320, or both.
8. The method of Embodiment 7, wherein the patient is up to two years old.
9. The method of any one of the preceding Embodiments, wherein the patient has a weight of at least 5 kg.
10. The method of any one of the preceding Embodiments, wherein the patient has a gestational age of at least 29 weeks.
11. The method of any one of the preceding Embodiments, wherein the patient exhibits AUC_{0-∞} of greater than 13.4 day·mg/mL.
12. The method of any one of the preceding Embodiments, the method comprising administering the dose at the beginning of the RSV season.
13. The method of any one of the preceding Embodiments, the method comprising administering the dose intramuscularly.
14. The method of any one of the preceding Embodiments, wherein the patient exhibits Down's Syndrome, an immunodeficiency, congenital lung disease, or congenital heart disease or a combination thereof.
15. The method of any one of the preceding Embodiments, wherein the patient exhibits congenital lung disease or congenital heart disease or both.
16. The method of any one of the preceding Embodiments, the method comprising administering the anti-RSV monoclonal antibody or an antigen binding fragment thereof in a composition comprising at least one of an ionic excipient, a buffer, a sugar, and a surfactant.
17. The method of Embodiment 16, wherein the ionic excipient comprises L-arginine hydrochloride at a concentration of 80 mM.
18. The method of Embodiment 16 or Embodiment 17, wherein the buffer comprises 30 mM L-histidine/L-histidine hydrochloride, the sugar comprises 120 mM sucrose, and/or the surfactant comprises polysorbate 80 in a range of 0.01% (w/v) to 0.05% (w/v).
19. The method of any one of Embodiments 17 to 19, wherein the anti-RSV monoclonal antibody or an antigen binding fragment thereof in the composition is stable at 2° C. to 8° C. for at least 3 months as determined by high performance size exclusion chromatography (HPSEC).
20. The method of any one of Embodiments 17 to 21, wherein the composition has a pH in a range of from 5.5 to 6.5.
21. The method of any one of Embodiments 17 to 21, wherein the the anti-RSV monoclonal antibody or an antigen binding fragment thereof is present in the composition at a concentration of 100 mg/mL.
22. The method of any one of the preceding Embodiments, wherein the anti-RSV monoclonal antibody or the antigen binding fragment of the anti-RSV monoclonal antibody comprises a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO:1 and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical SEQ ID NO:2.
   23. The method of any one of the preceding Embodiments, wherein the anti-RSV monoclonal antibody or the antigen binding fragment of the anti-RSV monoclonal antibody comprises a sequence comprising SEQ ID NO:1 and/or SEQ ID NO:2.
   24. The method of any one of the preceding Embodiments, wherein the anti-RSV monoclonal antibody or the antigen binding fragment of the anti-RSV monoclonal antibody comprises:
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence QASQDIVNYLN (SEQ ID NO:3);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence VASNLET (SEQ ID NO:4);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence QQYDNLPLT (SEQ ID NO:5);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence DYIIN (SEQ ID NO:6);
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence GIIPVLGTVHYGPKFQG (SEQ ID NO:7); and/or
   a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence ETALVVSETYLPHYFDN (SEQ ID NO:8).
25. The method of any one of the preceding Embodiments, wherein the anti-RSV monoclonal antibody or the antigen binding fragment of the anti-RSV monoclonal antibody comprises QASQDIVNYLN (SEQ ID NO:3), VASNLET (SEQ ID NO:4), QQYDNLPLT (SEQ ID NO:5), DYIIN (SEQ ID NO:6), GIIPVLGTVHYGPKFQG (SEQ ID NO:7), and/or ETALVVSETYLPHYFDN (SEQ ID NO:8).
26. The method of any one of the preceding Embodiments, wherein the anti-RSV monoclonal antibody or the antigen binding fragment of the anti-RSV monoclonal antibody comprises QASQDIVNYLN (SEQ ID NO:3), VASNLET (SEQ ID NO:4), QQYDNLPLT (SEQ ID NO:5), DYIIN (SEQ ID NO:6), GIIPVLGTVHYGPKFQG (SEQ ID NO:7), and ETALVVSETYLPHYFDN (SEQ ID NO:8).
27. The method of any one of the preceding Embodiments, wherein the anti-RSV monoclonal antibody comprises a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO:9 and/or a sequence which is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO:10.
28. The method of any one of the preceding Embodiments, wherein the anti-RSV monoclonal antibody comprises nirsevimab or wherein the antigen binding fragment of the anti-RSV monoclonal antibody comprises an antigen binding fragment of nirsevimab.

### Exemplary Pharmaceutical Unit Dose Embodiments

1. A pharmaceutical unit dose comprising 100 mg of nirsevimab, wherein said unit dose is suitable for intramuscular administration.
2. A pharmaceutical unit dose comprising 200 mg of nirsevimab, wherein said unit dose is suitable for intramuscular administration.
3. The pharmaceutical unit dose of any one of the preceding Embodiments, wherein the pharmaceutical unit dose comprising a composition comprising nirsevimab, the composition further comprising at least one of an ionic excipient, a buffer, a sugar, and a surfactant.
4. The pharmaceutical unit dose of Embodiment 3, wherein the ionic excipient comprises L-arginine hydrochloride at a concentration of 80 mM.
5. The pharmaceutical unit dose of Embodiment 3 or Embodiment 4, wherein the buffer comprises 30 mM L-histidine/L-histidine hydrochloride, the sugar comprises 120 mM sucrose, and the surfactant comprises polysorbate-80 in a range of 0.01% (w/v) to 0.05% (w/v).
6. The pharmaceutical unit dose of any one of Embodiments 3 to 5, wherein the nirsevimab in the composition is stable at 2° C. to 8° C. for at least 3 months as determined by high performance size exclusion chromatography (HPSEC).
7. The pharmaceutical unit dose of any one of Embodiments 3 to 6, wherein the composition has a pH in a range of from 5.5 to 6.5
8. The pharmaceutical unit dose of any one of the preceding Embodiments, wherein the nirsevimab is present at a concentration of 100 mg/mL.

### Exemplary Pharmaceutical Composition Embodiments

1. A pharmaceutical composition for the treatment or prevention of RSV infection, the composition comprising 100 mg of nirsevimab, wherein the composition is administered to a patient in the first year of life, the patient having a weight of at least 5 kg.
2. The pharmaceutical composition of Embodiment 1, wherein the composition is administered when the patient is entering their first RSV season.
3. A pharmaceutical composition for the treatment or prevention of RSV infection, the composition comprising 100 mg of nirsevimab, wherein the composition is administered to a patient entering their first RSV season.
4. A pharmaceutical composition for the treatment or prevention of RSV infection, the composition comprising 200 mg of nirsevimab, wherein the composition is administered to a patient in the second year of life.
5. The pharmaceutical composition of Embodiment 3, wherein the patient is entering their second RSV season.
6. A pharmaceutical composition for the treatment or prevention of RSV infection, the composition comprising 200 mg of nirsevimab, wherein the composition is administered to a patient entering their second RSV season.
7. The pharmaceutical composition of any one of the preceding Embodiments, wherein the patient exhibits congenital lung disease or congenital heart disease or both.
8. The pharmaceutical composition of any one of the preceding Embodiments, wherein the patient exhibits AUC_{0-∞} of greater than 13.4 day·mg/mL.
9. The pharmaceutical composition of any one of the preceding Embodiments, the pharmaceutical composition having a pH in a range of from 5.5 to 6.5.
10. The pharmaceutical composition of any one of the preceding Embodiments, the pharmaceutical composition further comprising at least one of an ionic excipient, a buffer, a sugar, and a surfactant.
11. The pharmaceutical composition of Embodiment 10, the pharmaceutical composition comprising L-arginine hydrochloride at a concentration of 80 mM.
12. The pharmaceutical composition of any Embodiment 10 or 11, wherein the buffer comprises 30mM L-histidine/L-histidine hydrochloride, the sugar comprises 120 mM sucrose, and the surfactant comprises polysorbate-80 in a range of 0.01% (w/v) to 0.05% (w/v).
13. The pharmaceutical composition of any one of the preceding Embodiments, wherein the nirsevimab in the pharmaceutical composition is stable at 2° C. to 8° C. for at least 3 months as determined by high performance size exclusion chromatography (HPSEC).
14. The pharmaceutical unit dose of any one of the preceding Embodiments, wherein the nirsevimab is present at a concentration of 100 mg/mL.

The present invention is illustrated by the following examples. It is to be understood that the particular examples, materials, amounts, and procedures are to be interpreted broadly in accordance with the scope and spirit of the invention as set forth herein.

### EXAMPLES

### Example 1-Phase 2b Study

This Example describes a Phase 2b study of nirsevimab. The primary objective of the study was to compare the efficacy of nirsevimab versus placebo for a reduction of medically attended RSV-confirmed lower respiratory tract infections (LRTI) during the first 150 days post dose (that is, Day 151; the duration of a typical 5-month RSV season). The secondary efficacy objective included a comparison of nirsevimab versus placebo for a reduction of hospitalizations due to confirmed RSV through Day 151. Other objectives included a comparison of the safety and tolerability of nirsevimab versus placebo and an evaluation of the pharmacokinetic (PK) and anti-drug antibody (ADA) response to nirsevimab.

The target serum concentration of 6.8 μg/mL for the Phase 2b Study was based on RSV challenge studies in cotton rats, a model that has been proven to be a reliable predictor of target concentrations and was used for dose selection of palivizumab (Synagis Summary of Product Characteristics, 2018). Selection of the 50-mg IM dose for the Phase 2b study was based on the population-PK model used to identify a dose that would maintain nirsevimab serum concentrations above 6.8 μg/mL throughout the RSV season. Nirsevimab met this exposure target (achieved in >95% subjects) and the primary endpoint in this study by achieving a statistically significant relative risk reduction (RRR) of medically attended RSV-confirmed LRTI when compared to placebo whilst demonstrating safety.

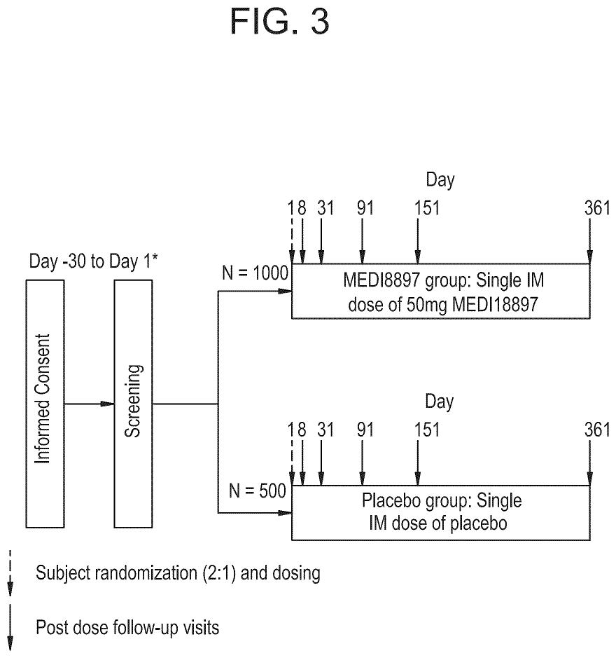

The Phase 2b Study was a randomized, double-blind, placebo-controlled study that evaluated the safety, efficacy, PK, and immunogenicity of nirsevimab in healthy preterm infants, born between 29 weeks 0 days and 34 weeks 6 days gestational age, entering their first RSV season (see FIG. 3). Subjects were not eligible for RSV prophylaxis with palivizumab based on the Joint Committee on Vaccination and Immunization, American Academy of Pediatrics, or other local or national guidelines, allowing for a placebo comparator group. Subjects were randomized at a 2:1 ratio to receive a single IM dose of 50 mg nirsevimab or placebo. Randomization was stratified by hemisphere (northern, southern) and by subject age at the time of randomisation (≤3 months, >3 to ≤6 months, >6 months). Subjects were followed for 360 days after dosing.

Subjects were monitored throughout the study for LRTI. All subjects seeking medical attention for a respiratory illness (in an inpatient or outpatient setting) were evaluated for the occurrence of LRTI. Subjects who had a primary hospitalisation for a respiratory illness, a respiratory deterioration during a hospitalisation, or who sought outpatient medical attention, including emergency room visits for a respiratory illness, were assessed for RSV by diagnostic testing of respiratory secretions and clinical assessment for the presence of LRTI. Testing for RSV was performed centrally using the US FDA-approved and Conformité Européenne-marked in vitro diagnostic real-time RT-PCR assay (Lyra RSV+hMPV assay, Quidel Corporation, San Diego, CA; www.quidel.com). A diagnosis of RSV LRTI required having a respiratory sample positive for RSV by the central RT-PCR assay.

### Subject Population

Disposition. As of the data cutoff (DCO) date for the primary analysis, all subjects were enrolled and a complete dataset for all randomised subjects was available through Day 151. Overall, 1,453 subjects (969 subjects, nirsevimab; 484 subjects, placebo) were randomised, with 1,447 subjects receiving a single dose of nirsevimab (968 subjects) or placebo (479 subjects; Table 1). Two subjects randomised to the placebo group incorrectly received nirsevimab; both subjects were included in the As-treated Population under the nirsevimab group.

The majority of subjects completed the Day 151 efficacy follow-up (948 subjects [97.8%], nirsevimab; 474 subjects [97.9%], placebo). As of the DCO date, 676 subjects (46.5%) completed the study and 710 (48.9%) were ongoing. The median number of days on study was 252 (range, 229 to 289 days).

Demographics. Demographic and baseline characteristics were comparable between the nirsevimab and placebo groups (Table 2). Overall, 52% of subjects were male, 72% were White, and 35% had siblings enrolled in the study.

### Efficacy

### Primary Endpoint—Incidence of Medically Attended RSV-Confirmed LRTI

Based on the primary analysis in the Intent-to-treat (ITT) Population, a single dose of 50 mg IM nirsevimab resulted in a relative risk reduction (RRR) in the incidence of medically attended RSV-confirmed LRTI through Day 151 of 70.2% (95% CI: 52.4%, 81.3%) when compared to placebo (p<0.0001; Table 3). Similar results were seen based on the same primary analysis model in the Per-protocol Population and the supporting Cochran-Mantel-Haenszel test in the ITT Population (Table 3).

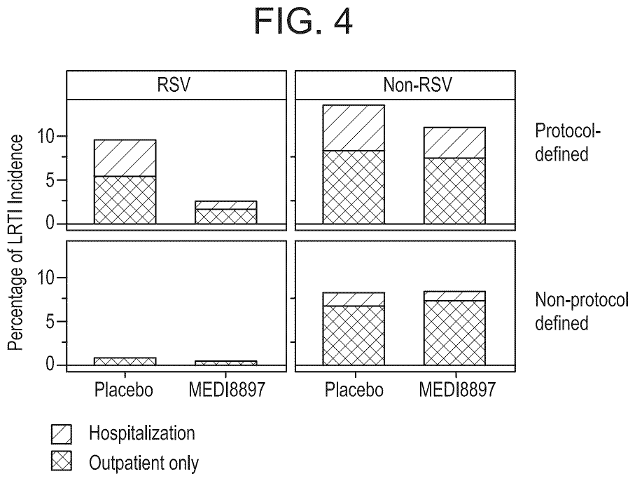

The incidence of all medically attended LRTI (protocol and non-protocol defined) through Day 151 was 19.7% in the nirsevimab group and 25.8% in the placebo group. As shown in Table 3, protocol-defined RSV-confirmed LRTI was seen in 2.6% of subjects in the nirsevimab group and 9.5% of subjects in the placebo group. The incidence of non-RSV LRTI (protocol or non-protocol defined) was generally similar between the groups (FIG. 4), suggesting that infection due to other viruses was not increased in the nirsevimab group.

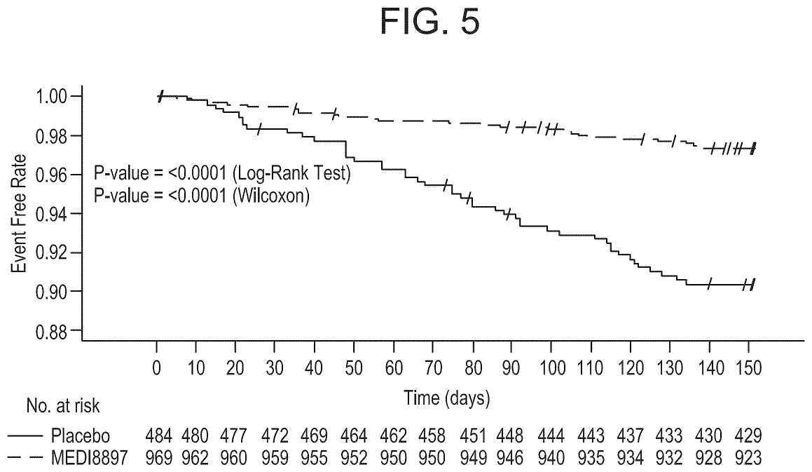

Efficacy of nirsevimab through Day 151 was confirmed with Kaplan-Meier analysis (p<0.0001; FIG. 5).

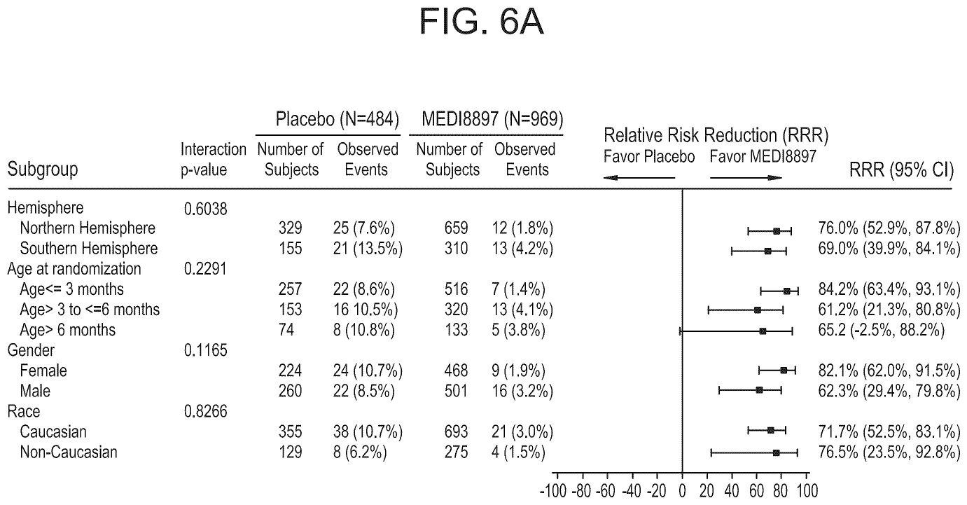

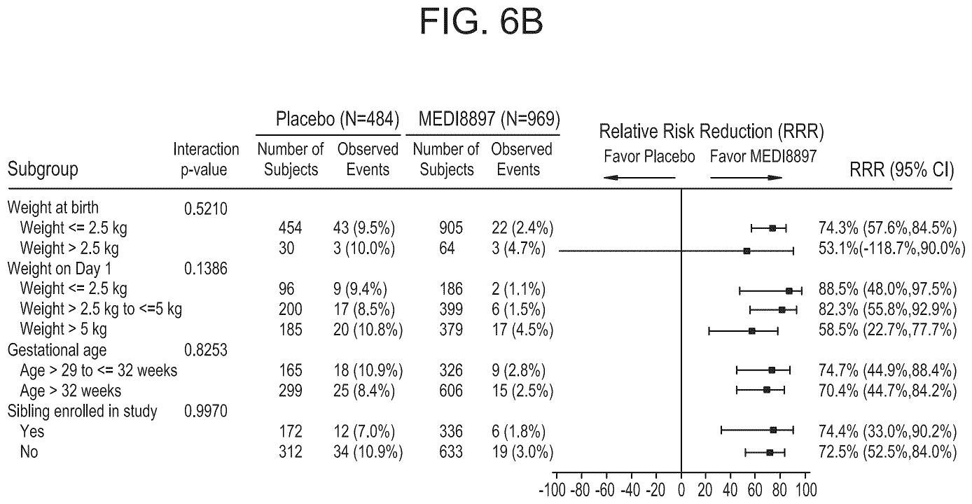

Subgroup Analysis. Subgroup analyses showed consistent results for hemisphere, age at randomisation, weight at birth, weight at Day 1, gestational age (GA), and siblings enrolled in the study, with no statistically significant interactions between each subgroup and treatment and relative risk reduction through Day 151 favouring nirsevimab vs placebo across all subgroups (FIG. 6). While efficacy was demonstrated for infants >5 kg, it was less than that seen for the smaller weight infants. Additional PK exposure-efficacy analyses showed that a dose of 100 mg would give similar exposures for infants ≥5 kg with a predicted improvement in efficacy.

TABLE 1
Subject Disposition, Primary Analysis

| Parameter | Placebo | | Nirsevimab | | Total | |
|---|---|---|---|---|---|---|
| Subjects randomised | 484 | | 969 | | 1453 | |
| Northern hemisphere | 329 | (68.0%) | 659 | (68.0%) | 988 | (68.0%) |
| Southern hemisphere | 155 | (32.0%) | 310 | (32.0%) | 465 | (32.0%) |
| Subjects treated ^{a} | 481 | (99.4%) | 966 | (99.7%) | 1447 | (99.6%) |
| Completed Day 151 efficacy | 474 | (97.9%) | 948 | (97.8%) | 1422 | (97.9%) |
| follow-up | | | | | | |
| Completed the study | 221 | (45.7%) | 455 | (47.0%) | 676 | (46.5%) |
| Early discontinuation | 27 | (5.6%) | 40 | (4.1%) | 67 | (4.6%) |
| Death | 4 | (0.8%) ^{b} | 2 | (0.2%) | 6 | (0.4%) |
| Lost to follow-up | 9 | (1.9%) | 17 | (1.8%) | 26 | (1.8%) |
| Withdrawal by parent/legal | 12 | (2.5%) | 19 | (2.0%) | 31 | (2.1%) |
| representative | | | | | | |
| Other | 2 | (0.4%) | 2 | (0.2%) | 4 | (0.3%) |
| Subjects who are ongoing | 236 | (48.8%) | 474 | (48.9%) | 710 | (48.9%) |

| | | | |
|---|---|---|---|
| Days on study as of DCO, | 252.0 | 251.5 | 252.0 |
| median (min, max) | (229, 289) | (229, 289) | (229, 289) |

DCO = data cutoff;
max = maximum;
min = minimum.
Denominator in the percentage calculation is based on the number of randomised subjects.
^{a} Two subjects randomised to the placebo group incorrectly received nirsevimab. Both subjects are included in the As-treated Population under the nirsevimab group.
^{b} One of the 4 deaths in the placebo group occurred after Day 361.

TABLE 2
Demographics and Baseline Characteristics—Intent-to-treat
Population

| | Placebo | | Nirsevimab | | Total | |
|---|---|---|---|---|---|---|
| Characteristic | (N = 484) | | (N = 969) | | (N = 1453) | |
| Age (months) at | 3.28 | (2.31) | 3.29 | (2.22) | 3.29 | (2.25) |
| randomisation, mean | | | | | | |
| (SD) | | | | | | |
| Weight (kg) on Day | 4.51 | (1.96) | 4.60 | (1.92) | 4.57 | (1.93) |
| 1, mean (SD) | | | | | | |
| Gender: Male | 260 | (53.7%) | 501 | (51.7%) | 761 | (52.4%) |

Race

| White | 355 | (73.3%) | 693 | (71.6%) | 1048 | (72.2%) |
|---|---|---|---|---|---|---|
| Black or African | 67 | (13.8%) | 189 | (19.5%) | 256 | (17.6%) |
| American | | | | | | |
| Asian | 10 | (2.1%) | 5 | (0.5%) | 15 | (1.0%) |
| American Indian or | 1 | (0.2%) | 0 | | 1 | (0.1%) |
| Alaskan | | | | | | |
| Native | | | | | | |
| Native Hawaiian or | 3 | (0.6%) | 8 | (0.8%) | 11 | (0.8%) |
| Other | | | | | | |
| Pacific Islander | | | | | | |
| Other | 43 | (8.9%) | 61 | (6.3%) | 104 | (7.2%) |
| Multiple categories | 5 | (1.0%) | 12 | (1.2%) | 17 | (1.2%) |
| checked | | | | | | |
| Gestational age | 32.7 | (1.5) | 32.7 | (1.4) | 32.7 | (1.4) |
| (weeks), mean (SD) | | | | | | |
| Gestational age >29 | 165 | (35.6%) | 326 | (35.0%) | 491 | (35.2%) |
| to <32 weeks | | | | | | |
| Gestational age >32 | 299 | (64.4%) | 606 | (65.0%) | 905 | (64.8%) |
| weeks | | | | | | |
| Siblings enrolled in | 172 | (35.5%) | 336 | (34.7%) | 508 | (35.0%) |
| the study | | | | | | |

SD = standard deviation.

TABLE 3
Incidence of Medically Attended RSV-confirmed LRTI Through 150
Days Post Dose

| | | | | |
|---|---|---|---|---|
| | | | Relative Risk | |
| | Placebo | Nirsevimab | Reduction | |
| Analysis | (N = 484) | (N = 969) | (95% CI) | P value |

Poisson regression with robust variance

| Observed events | 46 | (9.5%) | 25 (2.6%) | NA | |
|---|---|---|---|---|---|
| Subjects requiring | 9 | (1.9%) | 21 (2.2%) | NA | |
| imputation ^{a} | | | | | |
| Efficacy | | | | 70.2% (52.4%, | <0.0001 |
| | | | | 81.3%) | |

Stratified Cochran-Mantel-Haenszel test

| Observed events | 46 | (9.5%) | 25 (2.6%) | NA | |
|---|---|---|---|---|---|
| Efficacy | | | | 72.9% (56.5%, | <0.0001 |
| | | | | 83.1%) | |

CI = confidence interval;
LRTI = lower respiratory tract infection;
NA = not applicable;
RSV = respiratory syncytial virus.
^{c} Subjects who had no events and were not followed through 150 days post dose.

### Secondary Endpoint—Incidence of RSV LRTI Hospitalization

Based on the primary analysis model in the Intent-to-treat (ITT) Population, a single dose of 50 mg IM nirsevimab resulted in a relative risk reduction (RRR) in the incidence of RSV LRTI hospitalisation through Day 151 of 78.8% (95% CI: 52.3%, 90.6%) when compared to placebo (p=0.0002; Table 4). Similar results were seen based on the same primary analysis model in the Per-protocol Population and the supporting Cochran-Mantel-Haenszel test in the ITT Population (Table 4).

### Efficacy by RSV Subtype

Nirsevimab demonstrated activity against RSV A and RSV B subtypes (Table 5). RSV A and RSV B subtypes were responsible for a similar proportion of RSV LRTI overall and hospitalisation through Day 151. The incidence due to either subtype was notably lower in the nirsevimab group vs the placebo group.

TABLE 4
Incidence of RSV LRTI Hospitalisation Through 150 Days Post Dose

| | | | | |
|---|---|---|---|---|
| | Placebo | Nirsevimab | Relative Risk Reduction | |
| Analysis | (N = 484) | (N = 969) | (95% CI) | P value |

Poisson regression with robust variance

| Observed events | 20 | (4.1%) | 8 | (0.8%) | NA | |
|---|---|---|---|---|---|---|
| Subjects requiring imputation^{a} | 9 | (1.9%) | 21 | (2.2%) | NA | |
| Efficacy | | | | | 78.8% (52.3%, 90.6%) | 0.0002 |

Stratified Cochran-Mantel-Haenszel test

| Observed events | 20 | (4.1%) | 8 | (0.8%) | NA | |
|---|---|---|---|---|---|---|
| Efficacy | | | | | 80.0% (55.0%, 91.1%) | <0.0001 |

CI = confidence interval; LRTI = lower respiratory tract infection; RSV = respiratory syncytial virus.
^{a} Subjects who had no events and were not followed through 150 days post dose.

TABLE 5
Summary of Efficacy by RSV Subtype
Through 150 Days Post Dose

| | Placebo | | Nirsevimab | |
|---|---|---|---|---|
| Endpoint | (N = 484) | | (N = 969) | |
| Incidence of medically attended RSV- | 46 | (9.5%) | 25 | (2.6%) |
| confirmed LRTI | | | | |
| RSV A | 24 | (5.0%) | 11 | (1.1%) |
| RSV B | 22 | (4.5%) | 14 | (1.4%) |
| Incidence of RSV LRTI hospitalisation | 20 | (4.1%) | 8 | (0.8%) |
| RSV A | 12 | (2.5%) | 5 | (0.5%) |
| RSV B | 8 | (1.7%) | 3 | (0.3%) |

LRTI = lower respiratory tract infection; RSV = respiratory syncytial virus.

### Safety

### Summary of Adverse Events

In the As-treated Population, treatment-emergent adverse event (TEAE) rates for the nirsevimab group were generally comparable or lower than the placebo group across the TEAE categories (Table 6). Overall, 83.7% of subjects in the nirsevimab group and 83.9% of subjects in the placebo group had at least 1 TEAE. TEAEs ≤1 day post dose occurred in 2.5% of subjects in both groups. In comparison to the placebo group, the nirsevimab group had a lower incidence of TEAEs occurring ≤7 days post dose (15.2% vs 12.5%, respectively), TEAEs≥Grade 3 in severity (12.3% vs 7.4%, respectively), or TESAEs (16.7% vs 10.4%, respectively).

Five deaths were reported during the study through Day 361 as of the DCO for the primary analysis, including 2 subjects (0.2%) in the nirsevimab group and 3 subjects (0.6%) in the placebo group. One additional subject in the placebo group died on Day 367. None of these deaths were related to study treatment according to the investigator.

Overall, the incidence of treatment-related AEs (nirsevimab 2.3%, placebo 2.1%); adverse events of special interest (AESIs), including hypersensitivity, immune complex disease, and thrombocytopenia (nirsevimab 0.5%, placebo 0.6%); and NOCDs (nirsevimab 0.3%, placebo 0.8%) was low and generally comparable between the placebo and nirsevimab groups. TEAEs that involved the skin and subcutaneous tissues (including diaper rash) were collected as skin reactions, with a few exceptions for skin reactions that could be definitively diagnosed such as impetigo, varicella, and scabies. Skin reactions were reported in a similar percentage of subjects in both treatment groups (nirsevimab 28.2%, placebo 26.5%).

### Adverse Events

The TEAE profile was generally comparable between the nirsevimab and placebo groups (Table 7). Upper respiratory tract infection (URTI) was the most common TEAE in both groups (38.5% nirsevimab, 32.6% placebo). Similarly, within each age group (≤3 months, >3 to ≤6 months, or >6 months), the percentage of subjects with TEAEs overall or ≤1 day or ≤7 days post dose was comparable between the nirsevimab and placebo groups (Table 6, Table 8).

The percentage of subjects with treatment-related TEAEs according to the investigator was generally similar between the nirsevimab and placebo groups overall and across the 3 age groups (Table 6, Table 8). None of the treatment-related events were reported in more than 2 subjects.

### Serious Adverse Events

Generally, there was a trend for lower treatment-emergent serious adverse event (TESAE) rates in the nirsevimab group vs the placebo group (overall: 10.4% nirsevimab, 16.7% placebo; (Table 9). The most common TESAEs, based on the nirsevimab group, were bronchiolitis (2.1% nirsevimab, 4.2% placebo), lower respiratory tract infection (1.4% nirsevimab, 2.7% placebo), pneumonia (1.4% nirsevimab, 2.1% placebo), and bronchitis (1.2% nirsevimab, 2.3% placebo). None of the TESAEs were considered related to study treatment by the investigator (Table 6).

### Adverse Events of Special Interest

AESIs were reported in 5 subjects (0.5%) in the nirsevimab and 3 subjects (0.6%) in the placebo group (Table 10). All events were Grade 1 in severity. The TEAE of petechiae that was reported as an AESI was 1-day duration and was reported by the site investigator based on parental description. There were no laboratory assessments for the petechiae.

TABLE 6
Summary of Treatment-emergent Adverse Events

| | Placebo | | Nirsevimab | | Total | |
|---|---|---|---|---|---|---|
| Subjects^{a} with | (N = 479) | | (N = 968) | | (N = 1447) | |
| At least 1 event | 402 | (83.9%) | 810 | (83.7%) | 1212 | (83.8%) |
| At least 1 event occurring ≤1 | 12 | (2.5%) | 24 | (2.5%) | 36 | (2.5%) |
| day post dose | | | | | | |
| At least 1 event occurring ≤7 | 73 | (15.2%) | 121 | (12.5%) | 194 | (13.4%) |
| days post dose | | | | | | |
| At least 1 investigational product- | 10 | (2.1%) | 22 | (2.3%) | 32 | (2.2%) |
| related event | | | | | | |
| At least 1 event of > Grade 3 | 59 | (12.3%) | 72 | (7.4%) | 131 | (9.1%) |
| severity | | | | | | |
| Death (Grade 5 severity) | 3 | (0.6%)^{b} | 2 | (0.2%) | 5 | (0.3%) |
| At least 1 serious event | 80 | (16.7%) | 101 | (10.4%) | 181 | (12.5%) |
| At least 1 serious and/or >Grade | 92 | (19.2%) | 114 | (11.8%) | 206 | (14.2%) |
| 3 severity event | | | | | | |
| At least 1 investigational product- | 0 | | 0 | | 0 | |
| related serious event | | | | | | |
| At least 1 AESI | 3 | (0.6%) | 5 | (0.5%) | 8 | (0.6%) |
| At least 1 investigational product- | 3 | (0.6%) | 5 | (0.5%) | 8 | (0.6%) |
| related AESI | | | | | | |
| At least 1 skin reaction | 127 | (26.5%) | 273 | (28.2%) | 400 | (27.6%) |
| At least 1 investigational product- | 4 | (0.8%) | 9 | (0.9%) | 13 | (0.9%) |
| related skin reaction | | | | | | |
| At least 1 skin hypersensitivity | 3 | (0.6%) | 5 | (0.5%) | 8 | (0.6%) |
| reaction | | | | | | |
| At least 1 investigational product- | 3 | (0.6%) | 5 | (0.5%) | 8 | (0.6%) |
| related skin hypersensitivity reaction | | | | | | |
| At least 1 NOCD | 4 | (0.8%) | 3 | (0.3%) | 7 | (0.5%) |

| | | | |
|---|---|---|---|
| At least 1 investigational product- | 0 | 0 | 0 |
| related NOCD | | | |

AESI = adverse event of special interest; NOCD = new onset chronic disease.
^{d}Subjects were counted once for each category regardless of the number of events.
^{b}One additional death occurred in the placebo group after Day 361.

TABLE 7
Ten Most Common Treatment-emergent Adverse Events
Occurring in the Nirsevimab Group by Preferred Term

| | | | |
|---|---|---|---|
| Preferred Term | Placebo | Nirsevimab | Total |
| (MedDRA version 21.0) | (N = 479) | (N = 968) | (N = 1447) |

| | | | | | | |
|---|---|---|---|---|---|---|
| Subjects with at least | 402 | (83.9%) | 810 | (83.7%) | 1212 | (83.8%) |
| 1 event | | | | | | |
| Upper respiratory tract | 156 | (32.6%) | 373 | (38.5%) | 529 | (36.6%) |
| infection | | | | | | |
| Nasopharyngitis | 83 | (17.3%) | 140 | (14.5%) | 223 | (15.4%) |
| Gastroenteritis | 41 | (8.6%) | 105 | (10.8%) | 146 | (10.1%) |
| Rhinitis | 43 | (9.0%) | 102 | (10.5%) | 145 | (10.0%) |
| Pyrexia | 53 | (11.1%) | 94 | (9.7%) | 147 | (10.2%) |
| Bronchitis | 55 | (11.5%) | 93 | (9.6%) | 148 | (10.2%) |
| Bronchiolitis | 51 | (10.6%) | 89 | (9.2%) | 140 | (9.7%) |
| Diarrhoea | 45 | (9.4%) | 88 | (9.1%) | 133 | (9.2%) |
| Lower respiratory tract | 50 | (10.4%) | 82 | (8.5%) | 132 | (9.1%) |
| infection | | | | | | |
| Conjunctivitis | 36 | (7.5%) | 75 | (7.7%) | 111 | (7.7%) |

MedDRA = Medical Dictionary for Regulatory Activities.

TABLE 8
Treatment-emergent Adverse Events by Age and Time Post Dose

| | Placebo | | Nirsevimab | | Total | |
|---|---|---|---|---|---|---|
| Parameter | (N = 479) | | (N = 968) | | (N = 1447) | |
| Subjects with at | 402 | (83.9%) | 810 | (83.7%) | 1212 | (83.8%) |
| least 1 event | | | | | | |
| Age ≤3 months | 211/255 | (82.7%) | 423/517 | (81.8%) | 634/772 | (82.1%) |
| Age >3 to ≤6 months | 126/150 | (84.0%) | 272/320 | (85.0%) | 398/470 | (84.7%) |
| Age >6 months | 65/74 | (87.8%) | 115/131 | (87.8%) | 180/205 | (87.8%) |
| Subjects with at least | 12 | (2.5%) | 24 | (2.5%) | 36 | (2.5%) |
| 1 event occurring ≤1 | | | | | | |
| day post dose | | | | | | |
| Age ≤3 months | 2/255 | (0.8%) | 10/517 | (1.9%) | 12/772 | (1.6%) |
| Age >3 to ≤6 months | 7/150 | (4.7%) | 10/320 | (3.1%) | 17/470 | (3.6%) |
| Age >6 months | 3/74 | (4.1%) | 4/131 | (3.1%) | 7/205 | (3.4%) |
| Subjects with at least | 73 | (15.2%) | 121 | (12.5%) | 194 | (13.4%) |
| 1 event occurring ≤7 | | | | | | |
| days post dose | | | | | | |
| Age ≤3 months | 36/255 | (14.1%) | 58/517 | (11.2%) | 94/772 | (12.2%) |
| Age >3 to ≤6 months | 21/150 | (14.0%) | 42/320 | (13.1%) | 63/470 | (13.4%) |
| Age >6 months | 16/74 | (21.6%) | 21/131 | (16.0%) | 37/205 | (18.0%) |

TABLE 9
Ten Most Common Treatment-emergent Serious Adverse Events
Occurring in the Nirsevimab Group by Preferred Term

| Preferred Term | Placebo | | Nirsevimab | | Total | |
|---|---|---|---|---|---|---|
| (MedDRA version 21.0) | (N = 479) | | (N = 968) | | (N = 1447) | |
| Subjects with at least | 80 | (16.7%) | 101 | (10.4%) | 181 | (12.5%) |
| 1 serious event | | | | | | |
| Bronchiolitis | 20 | (4.2%) | 20 | (2.1%) | 40 | (2.8%) |
| Lower respiratory tract | 13 | (2.7%) | 14 | (1.4%) | 27 | (1.9%) |
| infection | | | | | | |
| Pneumonia | 10 | (2.1%) | 14 | (1.4%) | 24 | (1.7%) |
| Bronchitis | 11 | (2.3%) | 12 | (1.2%) | 23 | (1.6%) |
| Gastroenteritis | 4 | (0.8%) | 8 | (0.8%) | 12 | (0.8%) |
| Pneumonia viral | 2 | (0.4%) | 7 | (0.7%) | 9 | (0.6%) |
| Lower respiratory tract | 3 | (0.6%) | 5 | (0.5%) | 8 | (0.6%) |
| infection viral | | | | | | |
| Pyrexia | 1 | (0.2%) | 3 | (0.3%) | 4 | (0.3%) |
| Upper respiratory tract | 3 | (0.6%) | 3 | (0.3%) | 6 | (0.4%) |
| infection | | | | | | |

| | | | | | |
|---|---|---|---|---|---|
| Croup infectious | 0 | 2 | (0.2%) | 2 | (0.1%) |

MedDRA = Medical Dictionary for Regulatory Activities.

TABLE 10
Adverse Events of Special Interest by Age and Preferred Term

| Age Preferred Term | Placebo | | Nirsevimab | | Total | |
|---|---|---|---|---|---|---|
| (MedDRA version 21.0) | (N = 479) | | (N = 968) | | (N = 1447) | |
| Subjects with at least 1 AESI | 3 | (0.6%) | 5 | (0.5%) | 8 | (0.6%) |

Age ≤3 months

| | | | | | |
|---|---|---|---|---|---|
| Subjects with at least 1 event | 0/255 | 3/517 | (0.6%) | 3/772 | (0.4%) |
| Rash | 0/255 | 2/517 | (0.4%) | 2/772 | (0.3%) |
| Petechiae | 0/255 | 1/517 | (0.2%) | 1/772 | (0.1%) |

Age >3 to ≤6 months

| | | | | | | |
|---|---|---|---|---|---|---|
| Subjects with at least 1 event | 3/150 | (2.0%) | 1/320 | (0.3%) | 4/470 | (0.9%) |
| Rash | 2/150 | (1.3%) | 1/320 | (0.3%) | 3/470 | (0.6%) |

| | | | | | |
|---|---|---|---|---|---|
| Rash papular | 1/150 | (0.7%) | 0/320 | 1/470 | (0.2%) |

Age >6 months

| | | | | | |
|---|---|---|---|---|---|
| Subjects with at least 1 event | 0/74 | 1/131 | (0.8%) | 1/205 | (0.5%) |
| Rash macular | 0/74 | 1/131 | (0.8%) | 1/205 | (0.5%) |

AESI = adverse event of special interest; MedDRA = Medical Dictionary for Regulatory Activities.

### Pharmacokinetics

Following a single fixed 50 mg IM dose of nirsevimab, over 95% of measurable Day 151 nirsevimab serum concentrations were greater than the nonclinical EC₉₀ target of 6.8 μg/mL (Table 11).

Although the scheduled PK sampling scheme was sparse, AUC_{0-∞} and apparent t½ could be estimated by non-compartmental analysis for 26 infants with sufficient additional PK samples from unscheduled visits. Overall, the median AUC_{0-∞} and estimated apparent t½ were 5.3 day·mg/ml (range, 3.2 to 10.2 day·mg/mL) and 56.5 days (range, 46.8 to 81.1 days), respectively.

TABLE 11
Summary of Observed Nirsevimab Serum Concentrations by Scheduled
PK Sampling Timepoint - As-treated Population, Nirsevimab Subjects
with Available Serum Samples (Study D5290C00003)

| Parameter | Day 91 | Day 151 | | Day 361 | |
|---|---|---|---|---|---|
| (μg/mL) | (N = 881) | (N = 848) | | (N = 372) | |
| Mean (SD) | 36.0 (10.9) | 18.9 | (7.36) | 1.91 | (1.08) |
| Geomean (% CV) | 34.2 (30.3) | 17.5 | (38.9) | 1.64 | (56.3) |

| | | | |
|---|---|---|---|
| Range | 4.18, 71.9 | 2.24, 61.54 | 0.51, 6.21 |

CV = coefficient of variation; PK = pharmacokinetic; SD = standard deviation.
Includes data only from subjects in the nirsevimab group who had at least one measurable post-dose nirsevimab serum sample.

### Anti-Drug Antibody

Overall, the rate and titres of anti-drug antibodies (ADA) were low, and in ADA-positive subjects there was no effect on PK or safety. Of the subjects who had serum samples available for testing, ADA was detected post baseline in 4.3% (40/921) of subjects in the nirsevimab group and 2.8% (13/466) of subjects in the placebo group: on Day 91 (nirsevimab 1.2%, placebo 0.9%), Day 151 (nirsevimab 2.0%, placebo 1.4%), and Day 361 (nirsevimab 3.5%, placebo 1.5%). ADA titres ranged from 1:50 to 1:3,200 in the nirsevimab group and 1:50 to 1:200 in the placebo group.

Of the nirsevimab subjects who were post-baseline ADA positive, ADA targeting the YTE domain was observed in 4/17 subjects (23.5%) on Day 151 and 10/15 subjects (66.7%) on Day 361. Two nirsevimab subjects had NAb on Day 361. Of the placebo subjects who were post-baseline ADA positive, ADA targeting the YTE domain was observed in 3/3 subjects on Day 361; 1 subject had NAb on Day 361.

### Nirsevimab Resistance

All 103 RSV-positive serum samples (from 40 nirsevimab-dosed subjects and 63 placebo-dosed subjects; determined by the Lyra RSV+hMPV real-time RT-PCR assay) were subjected to Sanger sequencing of the F gene and resulted in complete full-length F gene sequence for analysis with 53 RSV A and 50 RSV B sequences. Of these samples, 84 (from 29 nirsevimab-dosed subjects and 55 placebo-dosed subjects) represented unique incidences of LRTI (protocol and non-protocol defined) that occurred within the first season (through Day 151). The remaining 19 sequences were isolated from samples that were collected in error from subjects with URTI (4 nirsevimab dosed, 3 placebo dosed), LRTI outside the 150-day window (6 nirsevimab dosed, 2 placebo dosed), or from multiple sample collections from the same subject during a single clinical incidence that resulted in identical sequences (1 nirsevimab dosed, 3 placebo dosed). All F gene sequences were aligned and compared to a consensus sequence to identify potential amino acid variations within the nirsevimab binding site (Table 12). Amino acid variation K209R in RSV A and I206M+Q209R in RSV B in the nirsevimab binding site have been seen in previous studies in subjects not dosed with study drug and have shown no change in nirsevimab susceptibility (Zhu et al, 2018 J Infect Dis. 218(4):572-580). Of the two other viruses identified with variations in the nirsevimab binding site compared to consensus, one encoded a N208S change in F sequence, and another isolate encoded changes at I64I/T+K68K/E+1206M+Q209R. The N208S change has previously been identified in the preclinical work, and this mutation in the context of RSV B9320 results in a substantial susceptibility shift to nirsevimab. The I64T+K68E+I206M+Q209R combination has previously not been naturally observed, and a recombinant RSV B9320 virus engineered to have those 4 mutations in the binding site showed a shift in nirsevimab susceptibility. These 2 viruses containing resistance-associated mutations were identified in nasal samples from nirsevimab-dosed subjects who were hospitalized for LRTI.

TABLE 12
Amino Acid Variation Within the Nirsevimab Binding Site of F and Susceptibility Profiles

| | Amino Acid | | | | | Fold |
|---|---|---|---|---|---|---|
| | Variations Identified | | | | Nirsevimab | Change to |
| RSV | in the Nirsevimab | | No of Sequences ^{a} | | IC₅₀ | Reference |
| Subtype | Binding Site | Subject ID | Placebo | Nirsevimab | (ng/mL) | Virus |
| RSV A | K209R | 20029290003 | 0 | 2 | 4.2 ^{b} | 0.8 ^{b} |
| | | 20029290004 | | | | |
| | N208S | 20029560006 | 0 | 1 | 54161 ^{c} | 24618 ^{c} |
| RSV B | I206M, Q209R | Multiple ^{d} | 12 | 5 | 0.4 ^{c} | 0.1 ^{c} |
| | I64I/T, K68K/E, | 20029540026 | 0 | 1 | >ULOQ | >72 ^{e} |
| | I206M, Q209R | | | | | |

IC50 = half-maximal inhibitory concentration;
ID = identification;
No = number;
RSV = respiratory syncytial vims;
ULOQ = upper limit of quantification.
^{e} Total number of RSV A and RSV B sequences were 53 and 50, respectively
^{f} Described in report ID8897-0013
^{g} Described in Zhu et al., 2018 J Infect Dis. 218(4):572-80.
^{h} Subject IDs: 20030680022, 20033400003, 20034020002, 20034470001, 20034150008, 20032830004, 20033140005, 20032860005, 20029480034, 20030780004, 20032760007, 20029980006, 20029980007, 20030680014, 20032820006, 20030070001, 20029600002.
ⁱ Recombinant virus constructed and tested in neutralization assay contained I64T + K68E + I206M + Q209R amino acids.

### Example 2

The results of the Phase 2b Study (see Example 1) demonstrated the clinical efficacy of a single 50-mg IM nirsevimab dose in preterm infants with gestational age ranging from 29 to <35 weeks. Further analysis determined that the fixed 50-mg dose evaluated in Example 1 resulted in a wide distribution of weight-normalized doses with an average of 13.2 mg/kg (interquartile range [IQR], 8.33, 17.2 mg/kg) and 83 unique dose levels amongst 968 infants that received nirsevimab. This broad distribution of dose levels facilitated exposure-response analyses without studying multiple fixed doses in this population or potentially error-prone, individualized weight-based doses.

Population-PK and exposure-response analyses were conducted with the following aims: 1) characterize relevant sources of inter-subject variability in nirsevimab PK in adults and premature infants, 2) explore the relationship between nirsevimab serum exposure and the primary efficacy outcome of medically attended RSV-confirmed LRTI, and 3) conduct dose-optimization analysis to identify clinical dose(s) for future studies.

### Study Data

The population pharmacokinetic (popPK) dataset included clinical serum pharmacokinetic (PK) data from the Phase 1 study in healthy adult volunteers (Griffin et al. 2017, Antimicrob Agents Chemother. 61(3), pii: e01714-16), the Phase 1b/2a study in healthy preterm infants 32 to <35 weeks gestational age (GA) (Domachowske et al. Pediatr Infect Dis J. 2018; 37(9):886-892), and the Phase 2b study in healthy preterm infants 29 to <35 weeks GA (Example 1). The exposure-response analysis dataset consists of the efficacy data from Phase 2b and includes the empirical Bayes estimates from the popPK analysis (based on 1530 concentrations from 102 adults and total 2348 concentrations from 988 infants with 43 and 134 concentrations below the limit of quantitation, respectively.)

### Sample and Data Collection

Details of the study designs, doses, and populations are outlined in Table 13.

TABLE 13
Summary of Clinical Studies Included in the PK Analysis Dataset

| Study # | Study Design/ | Population | Study Drug Dose | PK Sampling | |
|---|---|---|---|---|---|
| Phase/Status | Objective | (Npk; N BLQ) | and Route | Time | ADA |
| D5290C00001 | Design: Single | Healthy adults | IV: 300 mg | 0, 8 h, and on | Study Day 6 |
| Phase I | Ascending | 182 (1530: 43) | (n = 6), 1000 mg | Study Day 2, 4, | 31, 91, 181, and |
| Completed | Dose, R, DB, | | (n = 6) 3000 mg | 6, 8, 15 22, 31, | 361 |
| | PC | | (n = 6) | 61, 91, 121, | |
| | Objective: | | IM: 100 mg | 151, 181, 271, | |
| | Safety, PK | | (n = 6); 300 mg | and 361 | |
| | | | (n = 78) | | |
| D529QC0002 | Design; Single | Healthy preterm | IM: 10 mg | Study Day 0, 8, | Study Day 31, |
| Phase 1b/2a | Ascending | infants 32 to < | (a = 8), 25 mg | 31, 151, 361 | 151, and 361 |
| Completed | Dose, R, DB, | 35 weeks GA | (n = 31), 50 mg | | |
| | PC | entering their | (n = 32) | | |
| | Objective: | first RSV | | | |
| | Safety, PK | season ^{a} | | | |
| | | 71 (278; 53) | | | |
| D5290C00003 | Design Single | Healthy preterm | IM: 50 mg | Study Day 91, | Study Day 91, |
| Phase 2b Pivotal | Dose R, DB, PC | infants 29 to < | (n = 968) | 151, 361 | 151, and 361 |
| Completed b* | Objective: | 35 weeks GA | | | |
| | Safety, efficacy, | and ≤ 8 months | | | |
| | PK | of age entering | | | |
| | | their first RSV | | | |
| | | season ^{a} | | | |
| | | 917(2373; 84) | | | |

DB = double-blind; GA = gestational age; IM = intramuscular; IV = intravenous; PC = placebo controlled; PK = pharmacokinetic; R = randomised: RSV = respiratory syncytial virus.
^{a} Preterm infant population not eligible to receive palivizumab per local guidelines.
^{b} Results from the primary analysis are presented in the Briefrag Document Section 3.3.1; final analyses will be included in the updated Investigator's Brochure (planned early 2019).
^{c} Liquid formulation. All other studies used a lyophiliized formulation of the study drug

### Bioanalytical Methods

Nirsevimab concentrations in human serum samples were measured using a validated fluorescence ELISA method (with a Lower Limit of Quantification (LLOQ) value of 0.5 μg/mL. An electrochemiluminescent (ECL), solution-phase, bridging immunoassay was used for detection, confirmation, and titration of anti-drug antibody (ADA) in human serum.

### Dataset Preparation and Handling

PK and clinical efficacy data were assembled using SAS (SAS Institute Inc., Cary, NC, USA) and R (R Foundation for Statistical Computing, Vienna, Austria) software. Data definition table and specifications were used to implement SAS programs followed by validation and quality control to ensure data consistency, reliability, and accuracy for all original or derived variables. The popPK data included demographics related parameters, for example, as baseline and time-varying age and bodyweight, sex, ethnicity, race, gestational age, and geographical location. Derived parameters included postmenstrual age, estimated as the sum of the gestational and the chronological age. Imputation methods for missing baseline or post-baseline bodyweight have previously been described (Robbie et al. in References Section is “Robbie et al. 2013 Antimicrob Agents Chemother. 57:6147-53.)

The exposure-response dataset was assembled by merging a data-frame containing the estimated individual PK parameters output from the NONMEM program (Icon plc, Dublin, Ireland) such as baseline clearance, projected AUC_{inf}, AUC_{0-inf}, AUC₀₋₁₅₀, with the demographic dataset for Phase 2b study, using R software.

### Data Disposition

Serum PK data from all subjects dosed with nirsevimab in Phase 1 and Phase 1b/2a were included in the popPK analysis. For Phase 2b, 51 infants with no quantifiable post-dose PK were excluded. Consequently, only the remaining 917 subjects exposed to nirsevimab and the 479 infants from the placebo group were included in the exposure response analysis.

### Population Pharmacokinetics and Exposure-Response Modeling Strategy

### Population Pharmacokinetics: Structural and Statistical Model

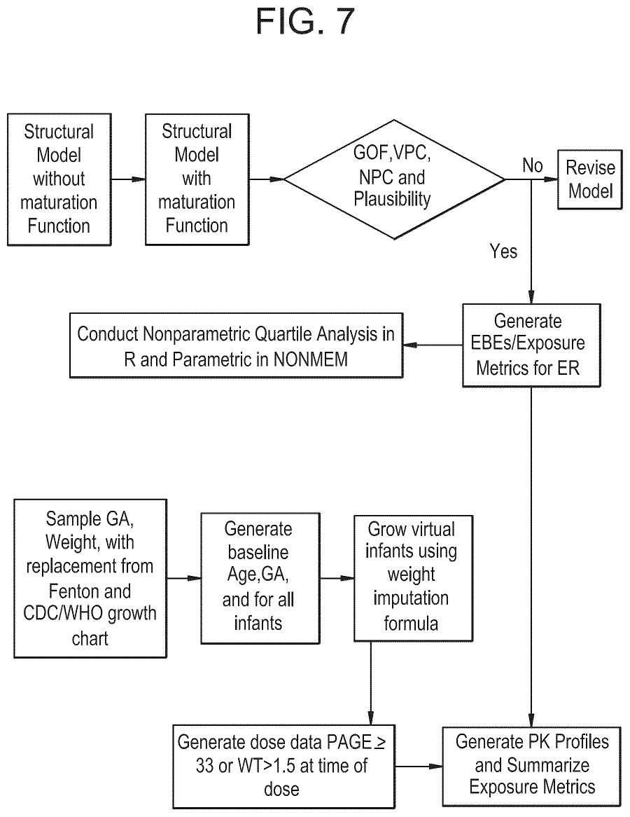

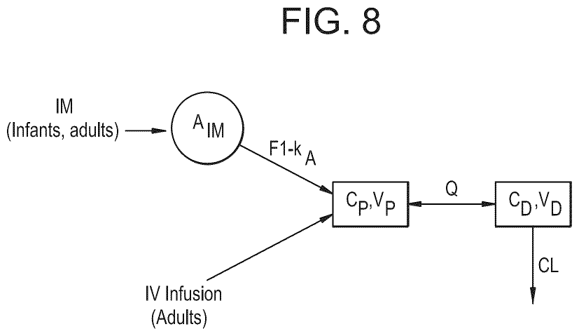

A two-compartment model with first order absorption, distribution clearance, as well as linear central elimination adequately characterized the PK data from the two studies. FIG. 7 and FIG. 8 show schematics of the popPK analysis work flow and the structural model, respectively.

Between subject variance (BSV) terms were estimated for systemic clearance, central volume of distribution, and absorption rate. Following allometric scaling of bodyweight on clearance and volume, the covariate model from the two studies only included the effects of bodyweight and maturation on clearance. Of note, the ETA distribution of clearance and absorption significantly deviated from the normality assumption for these parameters. Hence, a box-cox transformation was used to address this issue, which facilitated the simultaneous fitting of the densely sampled adult data and sparse PK data in infants. The strategy for the current work includes using the base estimate from the previous popPK model as the initial structural model for the analysis. The effects of allometrically scaled bodyweight, and maturation were assessed on clearance. Additional covariates such as anti-drug antibody on clearance, ethnicity on volume of distribution were also included in the covariate analysis. A full model approach followed by univariate elimination was used for final covariate selection. To maximize efficiency during parameter estimation, the PK parameters were mu-referenced, and Importance Sampling expectation maximization estimation algorithm was used within NONMEM. Details regarding the modeling methodology and workflow are outlined in FIG. 7.

### Model Evaluation

Standard diagnostics and goodness of fit (GOF) plots were used to assess the adequacy of the popPK model. The plausibility and precision of the PK parameter estimates were also used as criteria for model selection. Using Perl speaks NONMEM (PsN) (Lindbom et al., 2004 Comput Methods Programs Biomed. 75(2):85-94), nonparametric bootstrap of 500 sampled datasets with replacement stratified by population (for example, adults versus infants) were conducted to assess the robustness of the model and generate confidence interval of the parameter estimates. For further assessment of the model adequacy, simulation-based visual predictive checks were conducted using the final covariate model parameters. Similar methodology was adopted for the qualification and evaluation of the exposure response model.

### Exposure Metrics and Exposure-Response Analysis

The final covariate popPK model was used to generate empirical Bayesian estimates of the PK parameters for conducting the exposure-response (ER) analysis. Using R software, a cox proportional hazard model was fitted to the ER dataset to conduct semi-parametric exploratory quartile analysis for the primary efficacy outcome of Phase 2b (PCR-confirmed RSV positive medically-attended lower respiratory tract infection (MALRTI)).

Specifically, the incidence of the first MALRTI event was characterized using a time-to-event method. The survival function defined as the probability that an individual in the study experiences an event at time greater than 0. The probability of not having an MALRTI incident was estimated according to the following relationship shown in equation 1:

$$Surv ( t ) = exp ( - {\int }_{0}^{t} haz ( t ) dt ) \qquad ( 1 )$$

$$Survival at any time t = exponent ( - integral of instantaneous hazard dt ) .$$

The probability density function, which is also in this case the probability of MALRTI at any time t, was estimated as the product of the instantaneous hazard and the survival at time t as shown in equation 2.

$$pdf ( t ) = haz ( t ) * exp ( - {\int }_{0}^{t} haz ( t ) dt ) \qquad ( 2 )$$

The probability density function was determined for infants with an MALRTI incident at time=t while the survival function was used for infants with right censored efficacy data, for example, no known MALRTI incidence over the time of interest (150 days post dose), or discontinued study, or removed consent prior to the censored time. Notably, Schoenfeld residual plots were evaluated to assess the constant hazard assumption for the exploratory model over the 150-day observation period.

NONMEM was used to conduct a multivariate ER analysis to identify influential covariates affecting the risk of MALRTI in infants in Phase 2b study (Table 14). Various parametric distributions, such as Weibull, and Gompertz, were evaluated during base model development. The Laplacian method was used in NONMEM to determine the Objective function and the log likelihood function. The final model was bootstrapped, and visual predictive checks were generated and stratified by the relevant covariates.

TABLE 14
Covariates Screened in the Hazard Model

| Covariate | Type | Parameter Assessed |
|---|---|---|
| Projected | Continuous/ | Baseline hazard and Shape |
| AUC_{inf} | Categorical | (if Weibull) |
| Region | Categorical | Baseline hazard and Shape |
| | | (if Weibull) |
| Weight | Continuous | Baseline hazard and Shape |
| | | (if Weibull) |
| Postmenstrual | Continuous | Baseline hazard and Shape |
| Age | | (if Weiball) |

### Simulation Methodology

The final popPK model was used to predict nirsevimab serum PK in late term to term infants (GA>35, Phase 3 Study), and preterm infants as well as children with congenital heart and lung disease (GA<29, CHD/CLD, Phase 2/3). Baseline characteristics for the virtual infants were generated using random sampling with replacement from Fenton's growth chart (Fenton and Kim, 2013 BMC Pediatrics. 13:59-72) or Olsen et al. (2015 Pediatrics 135:e572-81) for premature infants and the CDC/WHO chart (available on the worldwide web at www.cdc.gov/growthcharts/who/girls_length_weight.htm) for the term to late term infants. Of note, the virtual infants were limited to a minimum of 36 weeks postmenstrual age at baseline to mimic the clinical course for preterm infants, that is, infants will not be dosed until clinically stable at 34 weeks or higher postmenstrual age in NICU. To capture the effects of maturation, time-varying bodyweight were determined using the previously described imputation algorithm for bodyweight. PK profile and exposure metrics were summarized and tabulated for virtual infants for a single RSV season for the Phase 3 population and two consecutive seasons for the Phase 2/3 population.

### Pharmacokinetic Results

### Patient Characteristics

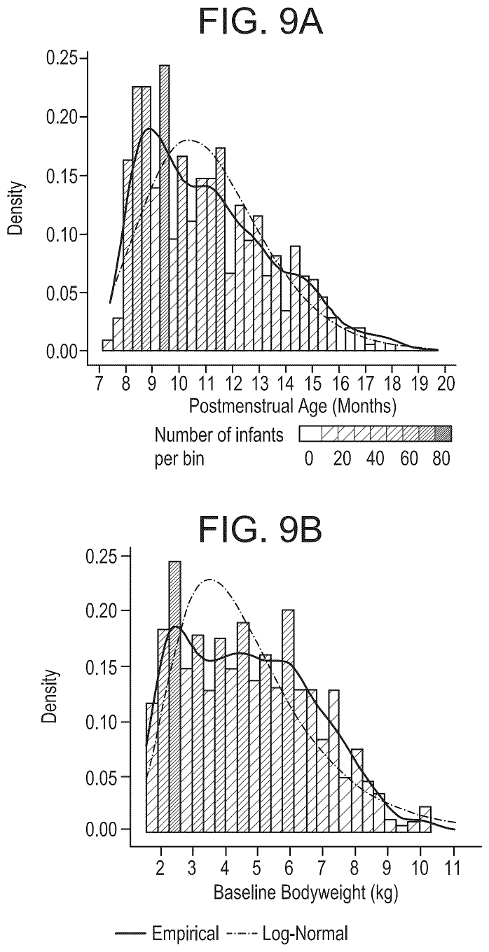

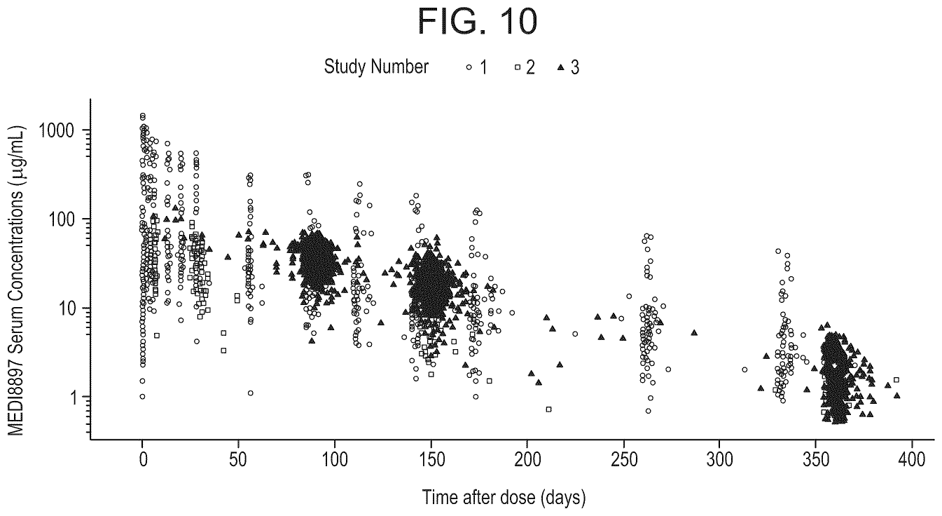

The popPK analysis dataset included 3881 serum PK observations from 1090 individuals. Baseline characteristics are outlined in Table 15. The median (range) baseline postnatal and postmenstrual age ranged from 3 months (0.1-11.9 months) and 13.9 months (7.4-19.7 months) respectively (FIG. 9A). Baseline bodyweight ranged from 1.6 kg to 11.1 kg with a median of 6.8 kg (FIG. 9B). This broad distribution of postnatal and postmenstrual age as well as bodyweight facilitated the characterization of size and maturation effects on PK parameters. Observed serum concentrations from all the studies are illustrated in FIG. 10 and Table 16

### PopPK Covariate Model

The effects of prematurity on PK parameters were modeled using a first-order exponential function as illustrated in equations 3-4:

$${CL}_{i} = CLpop * {( \frac{{WT}_{i}}{70} )}^{θ 1} * ( 1 - ( 1 - {β}_{CL} ) * \text{?} ) * {e}^{{η}_{CL}} \qquad ( 3 )$$

$${V}_{i} = Vpop * {( \frac{{WT}_{i}}{70} )}^{θ 2} * {e}^{{η}_{v}} \qquad ( 4 )$$

$$\text{?} \text{indicates text missing or illegible when filed}$$

Beta CL (β_{CL}) represents the fractional change in the clearance of a premature infant with respect to a term infant whilst the T50_{CL} denotes the corresponding maturation half-life of the parameter with respect to that of an adult. PAGEᵢ represents the sum of gestational age and postnatal age in months for each infant. Gestational age for adults was imputed to 40 weeks.

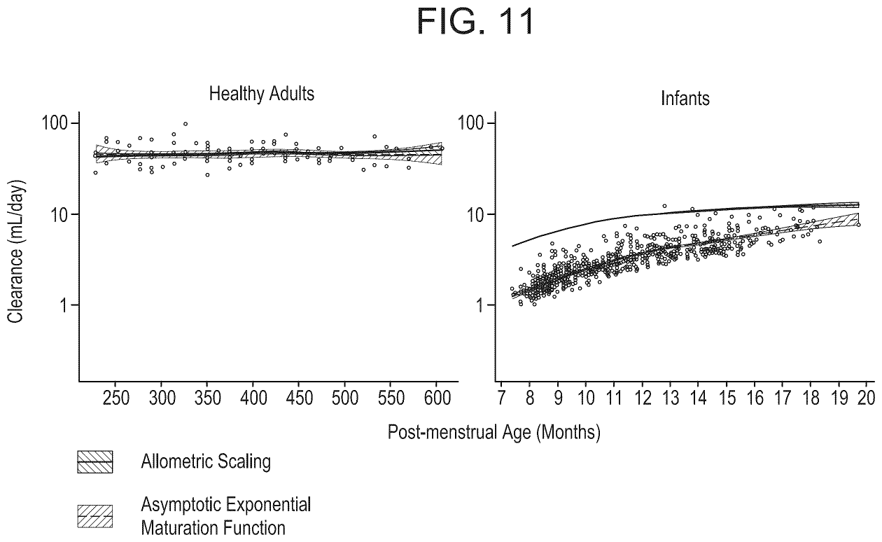

The final population PK covariate model parameters with bootstrap estimates are shown in Table 17. Effect of size and maturation on clearance are depicted in FIG. 11.

Since inclusion of maturation on the central volume of distribution did not improve model fitting, only allometric scaling remained on this parameter. Based on the model analysis, between subject variance (BSV) was less than 30% for both clearance and volume of distribution. Baseline and time-varying bodyweight, along with postmenstrual age were the most influential predictors of clearance.

### Model Evaluation

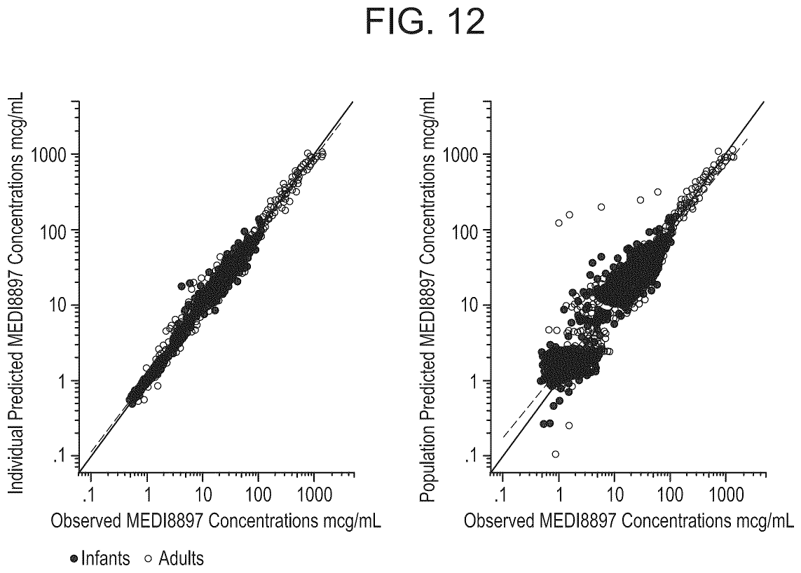

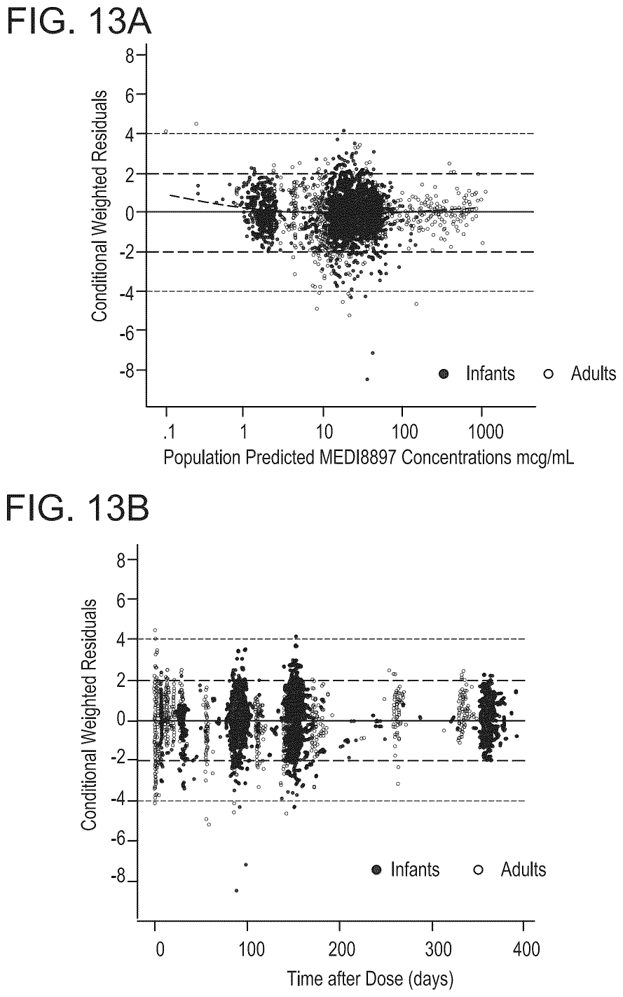

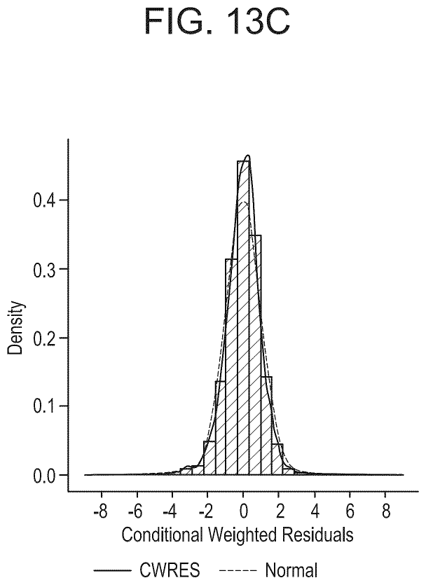

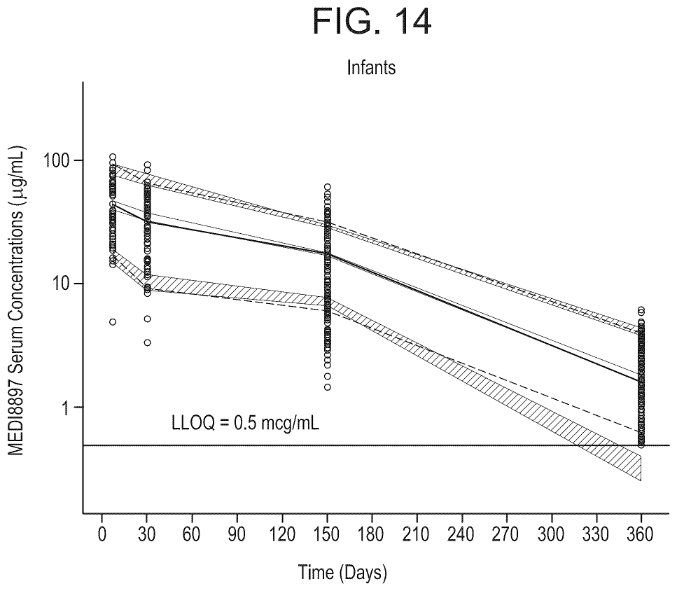

Inspection of the goodness of fit (FIG. 12, FIG. 13) and visual predictive check (FIG. 14) plots did not reveal any pertinent bias or misspecification in the model. The bootstrap results illustrate the stability of the final covariate model as all the bootstrapped datasets successfully converged. The 95% confidence interval (CI) of the bootstrap procedure included the median estimate of the original model for all parameters.

TABLE 15
Demographic and Baseline Characteristics of Subjects
Included in the Population PK Analysis Datasets

| | Study | | Study | | Study | | |
|---|---|---|---|---|---|---|---|
| | D5290C00001 | | D5290C00002 | | D5290C00003 | | |
| | N = 102 Adults | | N = 71 Infants | | N = 968 Infants | | p-value |
| Gestational Age (Weeks), | 40.0 | (0.00) | 33.1 | (0.78) | 32.7 | (1.42) | <0.001 |
| Mean (SD) | | | | | | | |
| Baseline Age (Months), | 377 | (94.6) | 6.50 | (2.64) | 3.27 | (2.20) | 0.000 |
| Mean (SD) | | | | | | | |
| Postmentrual Age | 387 | (94.6) | 14.3 | (2.64) | 10.9 | (2.22) | 0.000 |
| (Months), Mean (SD) | | | | | | | |
| Baseline Bodyweight (kg), | 78.0 | (14.9) | 6.81 | (1.89) | 4.59 | (1.91) | 0.000 |
| Mean (SD) | | | | | | | |
| Gender: Female, N (%) | 54 | (52.93%) | 42 | (59.2%) | 467 | (48.2%) | 0.155 |
| Racial Background, N (%): | | | | | | | |
| Native American | 0 | (0.00%) | 1 | (1.41%) | 0 | (0.00%) | |
| Asian | 0 | (0.00%) | 1 | (1.41%) | 5 | (0.52%) | |
| Black/African American | 56 | (54.9%) | 41 | (57.7%) | 187 | (19.3%) | |
| Caucasian | 46 | (45.1%) | 8 | (11.3%) | 693 | (71.7%) | |
| Hawarias | 0 | (0.00%) | 0 | (0.00%) | 8 | (0.83%) | |
| Multiple Race | 0 | (0.00%) | 2 | (2.82%) | 12 | (1.24%) | |
| Other | 0 | (0.00%) | 18 | (25.4%) | 62 | (6.41%) | |
| Outcome: Event N (%) | 0 | (%) | 0 | (30) | 25 | (2.58%) | |
| Excluded From PK | 0 | (0.00%) | 0 | (0.00%) | 55 | (5.68%) | 0.002 |
| Analysis, N (%) | | | | | | | |
| Ethnicity, N(%): | | | | | | | <0.001 |
| N/A | 0 | (0.00%) | 0 | (0.00%) | 1 | (0.10%) | |
| Hispanic | 3 | (2.94%) | 8 | (11.3%) | 225 | (23.230) | |
| Non-Hispanic | 99 | (97.1%) | 63 | (88.7%) | 742 | (76.7%) | |
| Treatment, N(%): | | | | | | | |
| MEDI8S97 10 mg IM | 0 | (0.00%) | 8 | (11.3%) | 0 | (0.00%) | |
| MEDI8897 50 mg IM | 0 | (0.00%) | 32 | (45.1%) | 968 | (100%) | |
| MEDI8897 25 mg IM | 0 | (0.00%) | 31 | (43.7%) | 0 | (0.00%) | |
| MEDI8S97 100 mg IM | 5 | (5.88%) | 0 | (0.00%) | 0 | (0.00%) | |
| MEDI8897 1000 mg IV | 6 | (5.88%) | 0 | (0.00%) | 0 | (0.00%) | |
| MEDI8897 300 mg IM | 78 | (76.5%) | 0 | (0.00%) | 0 | (0.00%) | |
| MEDI8897 300 mg IV | 6 | (5.88%) | 0 | (0.00%) | 0 | (0.00%) | |
| MEDI8897 3000 mg IV | 6 | (5.88%) | 0 | (0.00%) | 0 | (0.00%) | |

TABLE 16
Distribution of PK Samples from Infants Stratified by Study Visit

| | Study D5290C00002 | | Study D5290C00003 | | |
|---|---|---|---|---|---|
| | N = 278 | | N = 2268 | | p-value |
| Treatment: | | | | | |
| MEDI8897 10 MG IM | 29 | (10.4%) | N/A | | |
| MEDI8897 25 MG IM | 124 | (44.6%) | N/A | | |
| MEDI8897 50 mg IM | 125 | (45.0%) | 2268 | (100%) | |
| ADA Positive N(%) | 50 | (28.8%) | 84 | (3.70%) | |
| Hemisphere: | | | | | |
| Southern Hemisphere | 253 | (91.0%) | 895 | (39.5%) | <0.001 |
| Fraction LLOQ N (%) | 50 | (18.0%) | 101 | (4.45%) | <0.001 |
| PK Sample Visit: | | | | | <0.001 |
| Day 8 | 70 | (25.2%) | N/A | | |
| Day 31 | 70 | (25.2%) | N/A | | |
| Day 91 | 0 | (0.00%) | 904 | (39.9%) | |
| Day 151 | 70 | (25.2%) | 872 | (38.4%) | |
| Day 361 | 68 | (24.5%) | 425 | (18.7%) | |
| Unscheduled | 0 | (0.00%) | 07 | (2.95%) | |
| Excluded | 0 | (0.00%) | 38 | (1.68%) | 0.018 |

TABLE 17
Final Population PK Covariate Model Parameters with Bootstrap Estimates

| | | | Bootstrap | |
|---|---|---|---|---|
| | Estimate | BSV Estimate % CV | Estimate* | BSV Estimate |
| Parameter | (RSE %) | (RSE %) [Shrinkage %] | (95% CI) | 36CV (958%CI) |
| CL(L/day)** | 43.3 | 26.7 (13) [41] | 0.042 (0.04-0.045) | 28.9 (24.9-34.0) |
| Vp (L)** | 2990 | 24.3 (8) [13] | 3.08 (0.29-0.33) | 28.8 (22.6-35.7) |
| Q (L)** | 443 | | 0.44 (0.44-0.45) | |
| V_{D} (L)** | 1780 | | 1.72 (1.66-1.78) | |
| k_{A} (1/day) | 0.446 | 49 (21) [71] | 0.52 (0.44-0.59) | 58 (35.7-86.6) |
| F1 | 0.767 | | 0.75 (0.74-0.77) | |
| Box-cox CL Shape^{a} | 1.05 | | 1.16 (0.40-2.0)) | |
| c_{add} log scale | 0.205 | | 0.30 (0.43-0.10) | |
| Beta-CL | 0.292 | | 11.5 (7.5-17.2) | |
| T₅₀-CL (months) | 16.7 | | 0.60 (0.59-0.61) | |
| CLexp | 0.599 | | 0.89 (0.87-0.92) | |
| Vexp | 0.919 | | 0.27 (0.23-0.31) | |
| Box-cox k_{A} Shape^{a} | 0.752 | | 45.2% (22.5-62.2) | |
| Coff _{CL-Vp} | 54.3 (14.1%) | | | |
| Con _{Vp-kA} | 12.2 (126%) | | | |
| Corr _{CL-kA} | −10 (113%) | | | |

*A total of 500 bootstrap sampled datasets ran with a 95% convergence rate
^{a}Allometrically scaled to their respective exponent and a reference body weight of 70 kg
${( )}^{b} {η}_{trans , i} = ( \frac{{( {e}^{{η}_{i}} )}^{θ n} - 1}{θ n} ) where {η}_{i} and {θ}_{n} represents the untransformed between subject variance and the$ shape parameter, respectively.
% CV = sqrt(exp(ω²)−1);
Corr _{CL-Vp} = ω_{CL-Vp}/(sqrt(ω_{CL} * ω_{Vp}))

### Exposure-Response Results

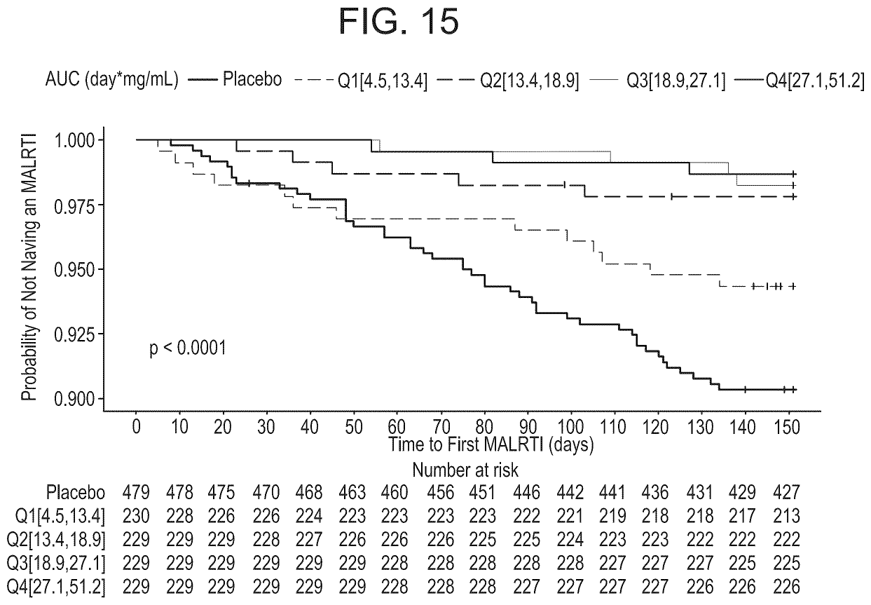

The results of the cox proportional hazard model revealed a positive relationship between nirsevimab exposure and the risk of medically-attended lower respiratory tract infection (FIG. 15). The constant hazard assumption was not violated for any of the covariates assessed in the model. The estimates of the final model and bootstrapped parameters are shown in Table 18.

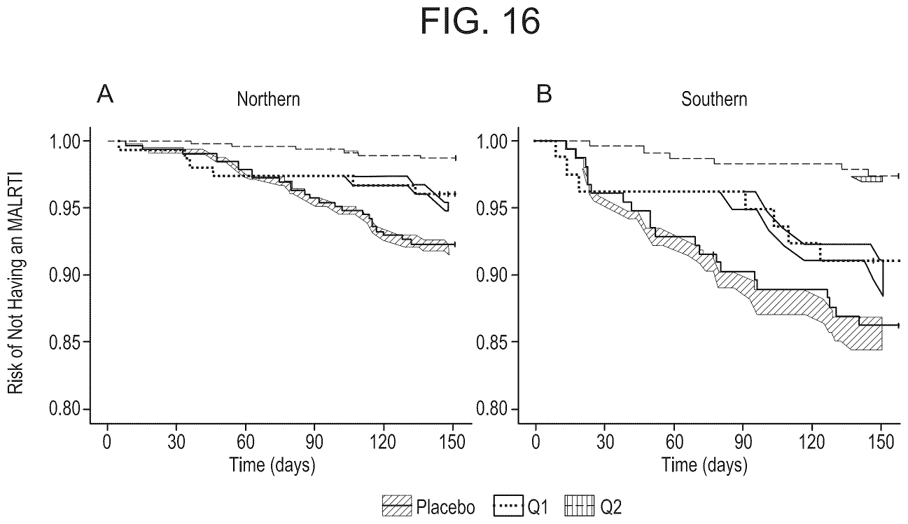

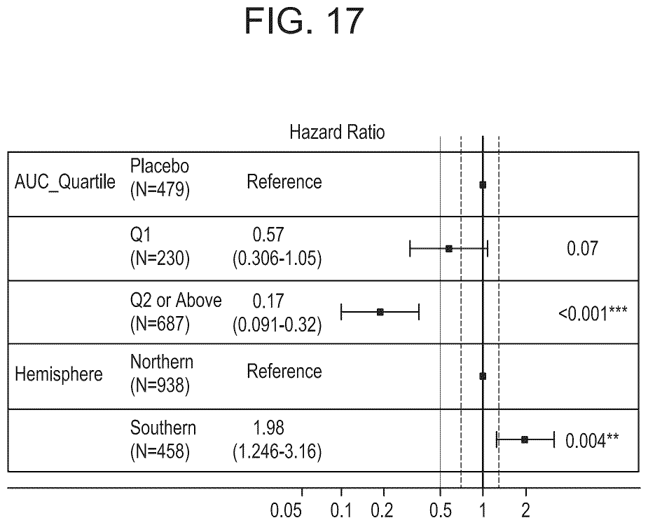

To further investigate these results, a parametric time-to-event analysis was conducted in NONMEM. An exponential hazard model adequately described the data as illustrated by the visual predictive check (VPC) (FIG. 16). The effects of geographical region and AUC quartiles on the risk of MALRTI were characterized and retained in the final covariate model (FIG. 17).

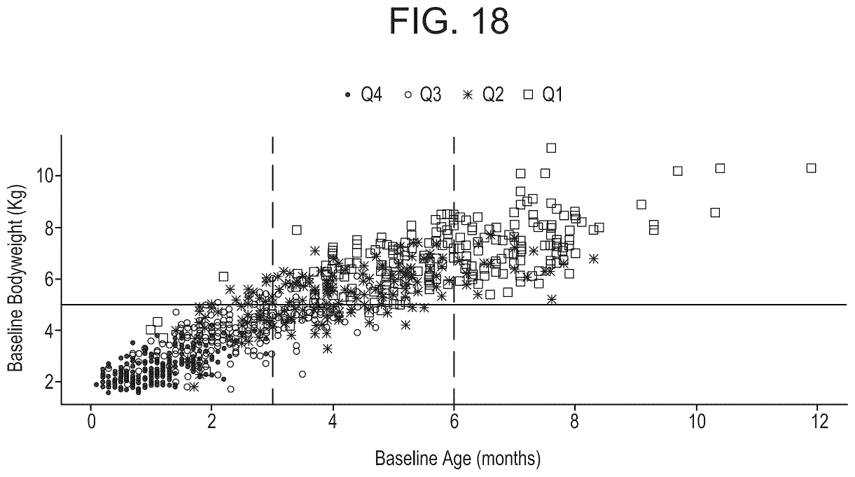

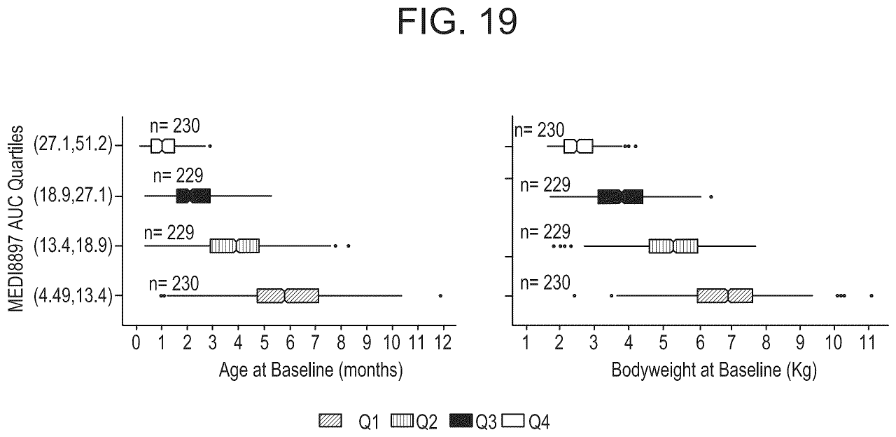

Given the marked effect of baseline bodyweight on clearance and consequently projected AUC_{inf}, a rank-based analysis was conducted using R software's Maxstat package to determine the optimal cut-point along the continuous distribution of body weight and weight-normalized doses to facilitate dose optimization for infant with suboptimal exposures (Hothorn and Lausen, 2003 Computational Statistics & Data Analysis. 43(2):525-55). A baseline bodyweight of 4.6 kg was determined to be the optimal weight cut-point from the analysis. In addition, exploratory analysis revealed a pertinent trend towards higher bodyweight for infants with Q1 serum AUC (FIG. 18, FIG. 19, & Table 19).

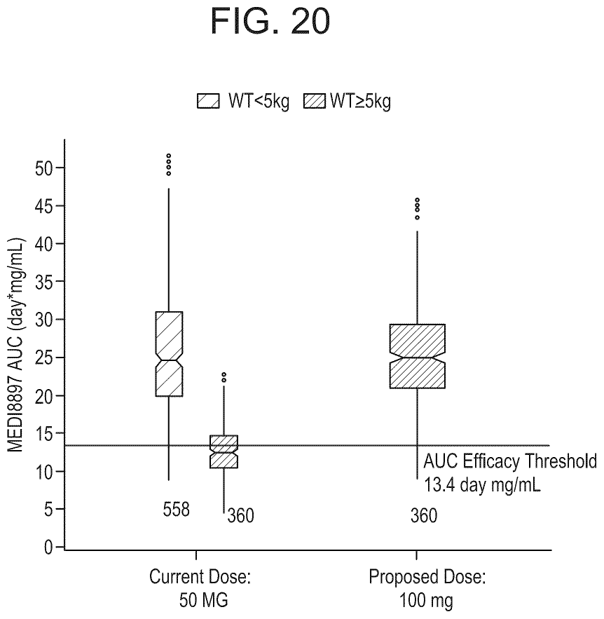

Only 40% of the infants weighing 5 kg or higher had serum AUC ≥Q2 compared to 97% of those weighing up to 5 kg. Infants with AUC ≥Q2 have a lower risk of MALRTI with a hazard ratio (HR) and 95% confidence interval (CI) of 0.17 (0.09, 0.32), compared to the infants who received placebo or had serum AUC below 13.4 day mg/mL. These results support a higher dose of 100 mg IM in infants with serum AUC below Q1 to match the exposure and consequently the benefit observed in infants with serum AUC ≥Q2 (FIG. 20).

TABLE 18
Parameter Table of the Final Model Estimate with Bootstrap

| | Model Estimate | Bootstrap Estimate |
|---|---|---|
| Description | (% RSE) | (95% CI) |
| Baseline hazard (1/day) | 0.00051 | 0.00052 |
| | (18.6) | (0.00034, 0.00072) |
| Lowest AUC Quartile (Q1) | −0.569 | −0.591 |
| effect on baseline hazard | (55.4) | (−1.33, −0.0103) |
| Region effect on baseline | 0.686 | 0.675 |
| hazard | (34.7) | (0.204, 1.14) |
| AUC > Q2 effect on baseline | −1.77 | −1.79 |
| hazard | (18.3) | (−2.58, −1.18) |

h(t) = λ₀*exp(β₁*AUC_Q1 + β₂*REGION + β₃*AUC_Q2)

TABLE 19
Summary of Baseline Characteristics Stratified by AUC Quartiles

| | Placebo | | Q1 | | Q2 | | Q3 | | Q4 | |
|---|---|---|---|---|---|---|---|---|---|---|
| | N = 479 | | N = 230 | | N = 229 | | N = 229 | | N = 229 | |
| WT (kg) | 4.51 | (1.96) | 6.83 | (1.30) | 5.25 | (1.06) | 3.71 | (0.95) | 2.52 | (0.56) |
| Cₘₐₓ (μg/mL) | 0.00 | (0.00) | 54.5 | (7.89) | 62.4 | (9.15) | 72.2 | (13.2) | 82.7 | (15.1) |
| Age (months) | 3.28 | (2.31) | 5.79 | (1.73) | 3.93 | (1.44) | 2.22 | (0.96) | 1.07 | (0.59) |
| PAGE (months) | 10.8 | (2.31) | 13.4 | (1.72) | 11.5 | (1.40) | 9.81 | (0.94) | 8.61 | (0.54) |

### Simulation

Baseline characteristics for virtual infants were simulated using data for term and preterm infants from the CDC/WHO growth charts and Olsen et. al. (Pediatrics. 2015; 135:e572-81). These data were included in the childSDS package v0.6.4 in R software. Firstly, the virtual infants' data were sampled with replacement from the 3^{rd} to the 97^{th} percentile body weight with gestational age ranging from 24 to 40 weeks with a minimum baseline bodyweight of 1.5 kg at the time of dose administration. Subsequently, the simulation data was assembled to mimic the expected population for the phase 3 trial (>35 GA) and phase 2/3 trial (<29 GA or term infants with CHD/CLD) dosed at up to 8 months postnatal age. The simulation results (Table 20) further support the proposed dose of 100 mg for infants over 5 kg in the first RSV season. Over 95% of infants are expected to reach serum AUC above the target threshold. The average Cₘₐₓ across all age groups are all within the safety margin or previously tolerated nirsevimab exposures. In addition, the PK parameters obtained from noncompartmental analysis of the simulated profiles are similar across age groups and doses, which further corroborates the adequacy of the exposure-matching approach for the proposed dosing strategy.

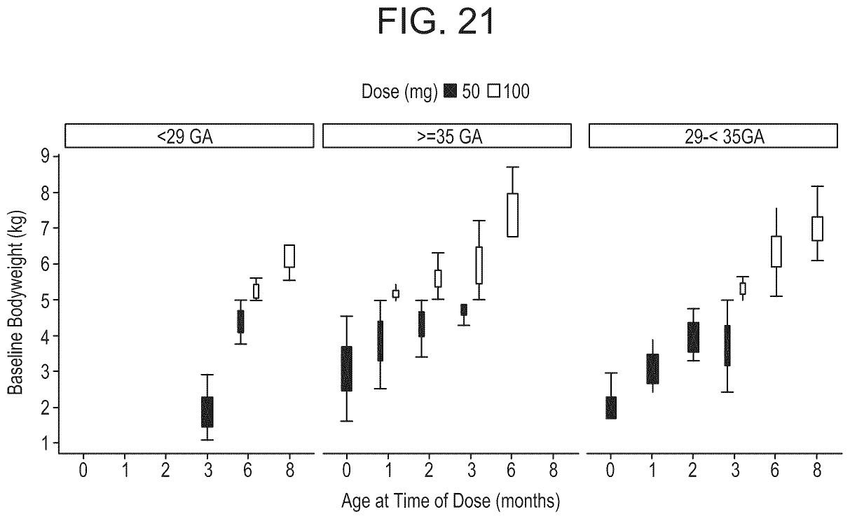

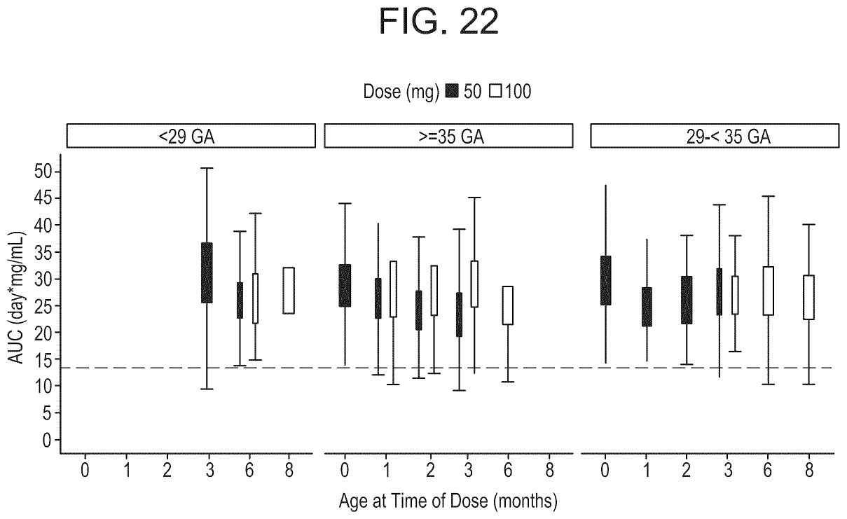

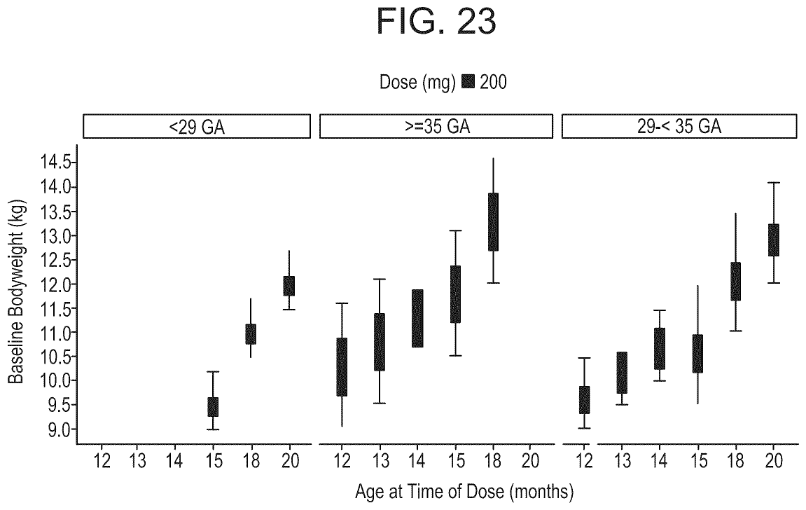

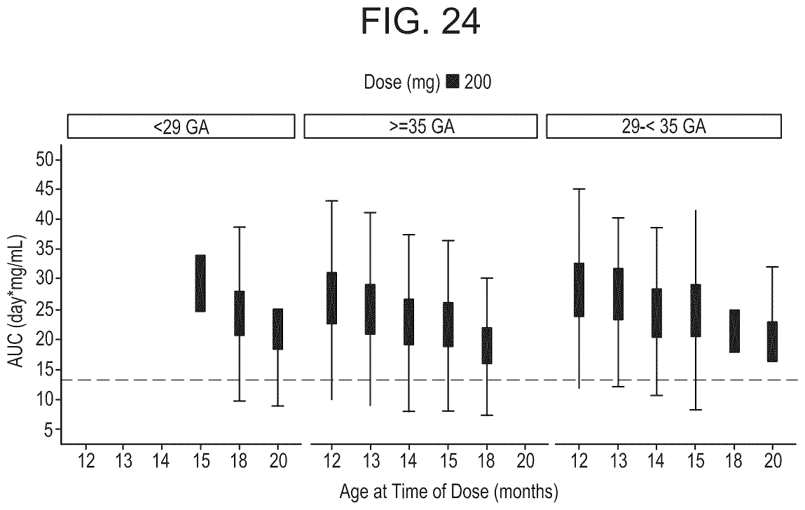

With the proposed doses shown in Table 20 and the anticipated body weight distributions in 1^{st} (FIG. 21) and 2^{nd} (FIG. 23) RSV season, it is predicted that the AUCs will be similar, largely overlapping and above the target AUC in majority of the infants (FIG. 22, FIG. 24).

### Conclusions

Based on the exposure-response analysis, a nirsevimab dose resulting in serum AUC above 13.4 day·mg/mL throughout the typical 5-month RSV season is anticipated to provide optimal protection against RSV in infants during the first year of life and high-risk children during the second year of life. Using the final popPK model, simulations indicate that a single fixed 50 mg dose (in infants <5 kg) or a 100 mg dose (in infants ≥5 kg) is expected to result in at least 80% of the population having an expected AUC_{0-∞} above 13.4 day·mg/mL in the first RSV season in the first year of life. Additionally, a single fixed 200-mg nirsevimab dose is expected to result in at least 80% of the population with expected AUC_{0-∞} being above 13.4 day·mg/mL and expected to be safe and effective for children in second year of life.

TABLE 20
Simulated Bodyweights and Exposures in the 1^{st} and 2^{nd} RSV Season

| | | <29 GA | | 29-<35 GA | | ≥35 GA | |
|---|---|---|---|---|---|---|---|
| | | N = 1234 | | N = 2905 | | N = 1993 | |
| 50 mg Dose | Bodyweight (kg) | 3.0 | (1.3) | 3.7 | (0.9) | 3.0 | (1) |
| (1^{st} RSV Season) | PAGE (months) | 8.4 | (1.5) | 8.8 | (1.2) | 9.6 | (1) |
| | Cₘₐₓ (μg/mL) | 191 | (82) | 173 | (63) | 155 | (55) |
| | Half-life (days) | 63.3 | (29) | 62.7 | (39) | 66.7 | (40) |
| | Projected AUC_{INF} | 27 | (24) | 26.5 | (24) | 25.7 | (24) |
| | % Above AUC Threshold: | 99 | | 99 | | 99 | |
| 100 mg Dose | Bodyweight (kg) | 6.0 | (0.5) | 6.5 | (1) | 6.5 | (0.7) |
| (1^{st} RSV Season) | PAGE (months) | 11.9 | (0.8) | 13.3 | (1.3) | 12.8 | (1.7) |
| | Cₘₐₓ (μg/mL) | 159 | (31) | 155 | (32) | 154 | (32) |
| | Half-life (days) | 63.9 | (26) | 62.6 | (22) | 66.6 | (40) |
| | Projected AUC_{INF} | 27 | (22) | 26 | (3) | 26 | (22) |
| | % Above AUC Threshold | 99 | | 99 | | 99 | |
| 200 mg Dose | Bodyweight (kg) | 10.8 | (1.1) | 11.3 | (1.3) | 11.4 | (1.2) |
| (2^{nd} RSV Season) | PAGE (months) | 21.6 | (2.2) | 22.9 | (2.6) | 22.8 | (2.1) |
| | Cₘₐₓ (μg/mL) | 235 | (62) | 221 | (57) | 226 | (62) |
| | Half-life (days) | <51 | (41) | 60 | (16) | 60 | (19) |
| | Projected AUC_{INF} | 24 | (26) | 22 | (26) | 23 | (27) |

| | | | |
|---|---|---|---|
| % Above AUC Threshold: | 97 | 95 | 95 |

### Example 3

Microneutralization assays were performed as follows: Briefly, 2-fold serial dilutions of MAb were introduced into 384-well microtiter plates in HEp-2 cell culture medium at a volume of 15 μL/well. Subsequently, 15 μL of either RSV A2 or RSV B 9320 virus diluted into HEp-2 cell culture medium to concentration ranging from 80 to 150 pfu/well and was added to each well including control wells containing HEp-2 cell culture medium alone, and plates were incubated for 1.5 hours at 37° C. with 5% CO2. HEp-2 cells were added at 2.5×10⁵ cells/mL in 30 μL to each well and the plates were incubated at 37° C. with 5% CO2. After 3 days for RSV A2 or 4 days for RSV B9320, medium was removed and 30 μL of ice cold 80% acetone/20% PBS was added to fix the cells.

Viral replication was measured by enzyme-linked immunosorbent assay (ELISA) using a horseradish peroxidase conjugated anti-RSV F MAb targeting the C site of RSV F (1331H) (Beeler and van Wyke Coelingh, J Virol. 63(7):2941-2950 (1989). 1331H MAb was diluted 1:6,000 in PBS and 30 μl was added to each well. Following two hours of incubation at 37° C., the plates were washed three times with PBS-T. TMB peroxidase 30 μL was added to each well and the plates were incubated at room temperature in the dark for 15 minutes. The reaction was stopped by the addition of 15 μL of 2N H2SO4 to each well. Substrate turnover was measured by monitoring absorbance at 450 nm using a microplate reader. IC50 values were calculated using a non-linear fit algorithm in Graphpad Prism using the log (inhibitor) vs. response with variable slope curve fit and represent the concentration of MAb required for a 50% reduction in absorbance measured at 450 nm.

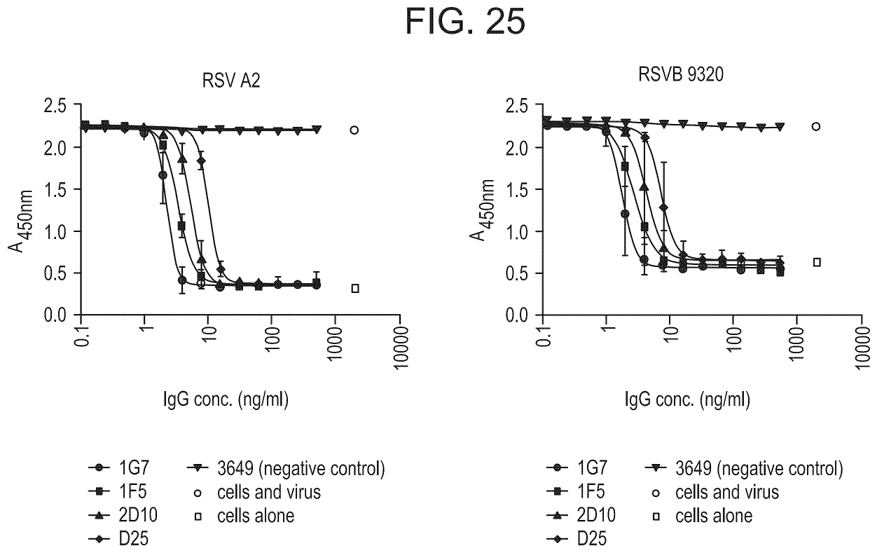

Results are provided in FIG. 25, which shows that nirsevimab, 1F5, 2D10, and D25 each inhibited RSV A2 and RSV B9320 replication in the microneutralization. The sequences of the variable regions of each of these antibodies are shown in Table 20A and Table 20B. Nirsevimab was the most effective, followed by 1F5, 2D10, and then D25.

TABLE 20A

| Light Chain Variable Region | |
|---|---|
| DIQMTQSPSS LSAAVGDRVT ITCQASQDIV | SEQ ID NO: 9 |
| NYLNWYQQKP GKAPKLLIYV ASNLETGVPS | |
| RFSGSGSGTD FSLTISSLQP EDVATYYCQQ | |
| YDNLPLTFGG GTKVEIK | |

TABLE 20B

| | Heavy Chain Variable Region | |
|---|---|---|
| D25 | QVQLVQSGAE VKKPGSSVMV SCQASGGPLR NYIINWLRQA PGQGPEWMGG | SEQ ID NO: 13 |
| | IIPVLGTVHY APKFQGRVTI TADESTDTAY IHLISLRSED TAMYYCATET | |
| | ALVVSTTYLP HYFDNWGQGT LVTVSS | |
| Nirsevimab | QVQLVQSGAE VKKPGSSVMV SCQASGGLLE DYIINWVRQA PGQGPEWMGG | SEQ ID NO: 10 |
| | IIPVLGTVHY GPKFQGRVTI TADESTDTAY MELSSLRSED TAMYYCATET | |
| | ALVVSETYLP HYFDNWGQGT LVTVSS | |
| 1F5 | QVQLVQSGAE VKKPGSSVMV SCQASGGPLE DYIINWVRQA PGQGPEWMGG | SEQ ID NO: 14 |
| | IIPVLGTVHY GPKFQGRVTI TADESTDTAY MELSSLRSED TAMYYCATET | |
| | ALVVSTTYLP HYFDNWGQGT LVTVSS | |
| 2D10 | QVQLVQSGAE VKKPGSSVMV SCQASGGPLE DYIINWVRQA PGQGPEWMGG | SEQ ID NO: 15 |
| | IIPVLGTVHY GPKFQGRVTI TADESTDTAY MELSSLRSED TAMYYCATET | |
| | ALVVSTTYRP HYFDNWGQGT LVTVSS | |

### Example 4

The cotton rat model was used as described in WO 2015/011391. Variants of nirsevimab (1G7-GLM, B12-1, E3-5, and E9-2) were made to alter the isoelectric point (pI) by incorporating germline residues in the heavy chain of the antibody. Sequences of the heavy chain variable regions of those variants are shown in Table 21; residues designated with a “˜” represent modifications made relative to nirsevimab.

The concentrations of human IgG in cotton rat serum samples on the day of lung harvest were determined using an ELISA method. The human antibodies were captured by a goat anti-human antibody bound to microtiter plates. The goat anti-human IgG (H+L) antibody (0.5 μg/mL in 1× PBS) was coated onto Nunc Maxisorp 384 well microtiter plates overnight at 4° C. in a 30 μL volume. Plates were washed then blocked with 60 μL of a solution of PBS+3% heat inactivated goat serum for 1 hour at room temperature. The blocking buffer was removed and samples were applied as follows: A two-fold serial dilution of the standard human antibody diluted in assay buffer was used for the standard curve with a concentration range of 500 ng/ml to 0.488 ng/ml. Standard curves were fitted using a 4 parameter curve fit.

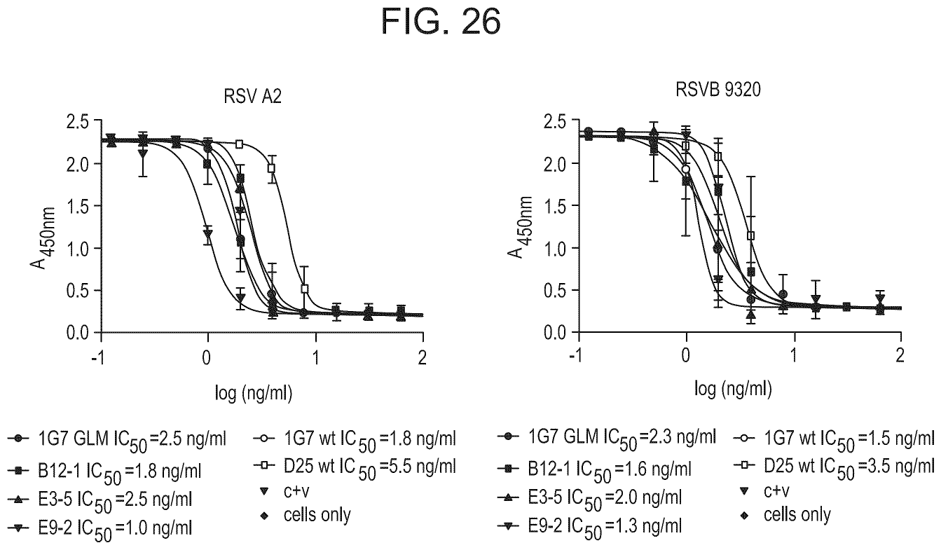

Results are provided in FIG. 26, showing that the variants described herein have lower IC50s than D25 in neutralizing both RSV A2 and RSV B9320. This also demonstrates that there was no loss of activity against the A2 or B9320 virus with increased activity seen with E9-2 and B12-1 and only a nominal loss of activity against the B9320 virus with 1G7 GLM and E3-5.

TABLE 21

| | | |
|---|---|---|
| 1G7-GLM | 1G7-GLM (pI 7.31) | SEQ ID NO: 16 |
| | ~ ~ | |
| | QVQLVQSGAEVKKPGSSVKVSCKASGGLLEDYIINWVRQAPGQGPEWMGGIIPVLG | |
| | ~ ~ ~ | |
| | TVHYGPKFQGRVTITADESTSTAYMHLSSLRSEDTAMYYCARETALVVSTTYLPHY | |
| | FDNWGQGTLVTVSS | |
| B12-1 | B12-1 (pI 6.97) | SEQ ID NO: 17 |
| | ~ ~ | |
| | QVQLVQSGAEVKKPGSSVKVSCKASGGLLEDYIINWVRQAPGQGPEWMGGIIPVLG | |
| | ~ | |
| | TVHYGPKFQGRVTITADESTDTAYMHLSSLRSEDTAMYYCATETALVVSTTYLPHY | |
| | FDNWGQGTLVTVSS | |
| E3-5 | E3-5 (pI 6.94) | SEQ ID NO: 18 |
| | ~ ~ | |
| | QVQLVQSGAEVKKPGSSVKVSCKASGGLLEDYIINWVRQAPGQGPEWMGGIIPVLG | |
| | ~ | |
| | TVHYGPKFQGRVTITADESTSTAYMELSSLRSEDTAMYYCATETALVVSTTYLPHY | |
| | FDNWGQGTLVTVSS | |
| E9-2 | E9-2 (pI 7.13) | SEQ ID NO: 19 |
| | ~ ~ | |
| | QVQLVQSGAEVKKPGSSVKVSCKASGGLLEDYIINWVRQAPGQGPEWMGGIIPVLG | |
| | ~ ~ | |
| | TVHYGPKFQRVTITADESTSTAYMHLSSLRSEDTAMYYCATETALVVSTTYLPHY | |
| | FDNWGQGTLVTVSS | |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1.-20. (canceled)

21. A pharmaceutical unit dose comprising an anti-Respiratory Syncytial Virus (RSV) monoclonal antibody or antigen-binding fragment thereof, wherein the anti-RSV monoclonal antibody or antigen-binding fragment is present in the pharmaceutical unit dose at a concentration of 100 mg/ml and at a dose of 50 mg, 100 mg, or 200 mg; and wherein the anti-RSV monoclonal antibody or antigen-binding fragment comprises
a light chain variable region comprising a CDR-L1 of SEQ ID NO: 3, a CDR-L2 of SEQ ID NO: 4, and a CDR-L3 of SEQ ID NO: 5, and
a heavy chain variable region comprising a CDR-H1 of SEQ ID NO: 6, a CDR-H2of SEQ ID NO: 7, and a CDR-H3 of SEQ ID NO: 8.

22. The pharmaceutical unit dose of claim 21, wherein the anti-RSV monoclonal antibody or antigen-binding fragment comprises a light chain variable region comprising SEQ ID NO: 9 and a heavy chain variable region comprising SEQ ID NO: 10.

23. The pharmaceutical unit dose of claim 21, wherein the anti-RSV monoclonal antibody is nirsevimab.

24. The pharmaceutical unit dose of claim 21, wherein the dose is 100 mg.

25. The pharmaceutical unit dose of claim 21, wherein the dose is 200 mg.

26. The pharmaceutical unit dose of claim 21, further comprising an ionic excipient, a buffer, a sugar, and a surfactant.

27. The pharmaceutical unit dose of claim 26, wherein:
the ionic excipient comprises 50 mM to 150 mM arginine or lysine,
the buffer comprises 20 mM to 50 mM histidine,
the sugar comprises 100 mM to 140 mM sucrose, and
the surfactant comprises 0.01% (w/v) to 0.05% (w/v) polysorbate-80;
wherein the pharmaceutical unit dose has a pH of 5.5 to 6.5.

28. The pharmaceutical unit dose of claim 26, wherein:
the ionic excipient comprises 80 mM L-arginine hydrochloride,
the buffer comprises 30 mM L-histidine, L-histidine hydrochloride, or a combination thereof,
the sugar comprises 120 mM sucrose, and
the surfactant comprises 0.02% (w/v) or 0.04% (w/v) polysorbate-80;
wherein the pharmaceutical unit dose has a pH of 5.5 to 6.5.

29. The pharmaceutical unit dose of claim 26, wherein the anti-RSV monoclonal antibody or antigen-binding fragment is stable in the pharmaceutical unit dose at 2° C. to 8° C. for at least 3 months, as determined by high performance size exclusion chromatography.

30. The pharmaceutical unit dose of claim 21, wherein the anti-RSV monoclonal antibody is nirsevimab, the dose is 50 mg, and the unit dose further comprises:
80 mM L-arginine hydrochloride,
30 mM L-histidine, L-histidine hydrochloride, or a combination thereof,
120 mM sucrose, and
0.02% (w/v) polysorbate-80;
wherein the pharmaceutical unit dose has a pH of 6.0.

31. The pharmaceutical unit dose of claim 21, wherein the anti-RSV monoclonal antibody is nirsevimab, the dose is 100 mg, and the unit dose further comprises:
80 mM L-arginine hydrochloride,
30 mM L-histidine, L-histidine hydrochloride, or a combination thereof,
120 mM sucrose, and
0.02% (w/v) polysorbate-80;
wherein the pharmaceutical unit dose has a pH of 6.0.

32. The pharmaceutical unit dose of claim 21, wherein the anti-RSV monoclonal antibody is nirsevimab, the dose is 200 mg, and the unit dose further comprises:
80 mM L-arginine hydrochloride,
30 mM L-histidine, L-histidine hydrochloride, or a combination thereof,
120 mM sucrose, and
0.02% (w/v) polysorbate-80;
wherein the pharmaceutical unit dose has a pH of 6.0.


SEQUENCE LISTING (19 sequences)

SEQ ID NO: 1 - protein, 214 aa, synthetic construct
  REGION 1..214: note = nirsevimab light chain
DIQMTQSPSSLSAAVGDRVTITCQASQDIVNYLNWYQQKPGKAPKLLIYVASNLETGVPSRFSGSGSGTDFSLTISSLQPEDVATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 456 aa, synthetic construct
  REGION 1..456: note = nirsevimab heavy chain
QVQLVQSGAEVKKPGSSVMVSCQASGGLLEDYIINWVRQAPGQGPEWMGGIIPVLGTVHYGPKFQGRVTITADESTDTAYMELSSLRSEDTAMYYCATETALVVSETYLPHYFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 11 aa, synthetic construct
  REGION 1..11: note = nirsevimab light chain CDR-L1
QASQDIVNYLN

SEQ ID NO: 4 - protein, 7 aa, synthetic construct
  REGION 1..7: note = nirsevimab light chain CDR-L2
VASNLET

SEQ ID NO: 5 - protein, 9 aa, synthetic construct
  REGION 1..9: note = nirsevimab light chain CDR-L3
QQYDNLPLT

SEQ ID NO: 6 - protein, 5 aa, synthetic construct
  REGION 1..5: note = nirsevimab heavy chain CDR-H1
DYIIN

SEQ ID NO: 7 - protein, 17 aa, synthetic construct
  REGION 1..17: note = nirsevimab heavy chain CDR-H2
GIIPVLGTVHYGPKFQG

SEQ ID NO: 8 - protein, 17 aa, synthetic construct
  REGION 1..17: note = nirsevimab heavy chain CDR-H3
ETALVVSETYLPHYFDN

SEQ ID NO: 9 - protein, 107 aa, synthetic construct [sequence omitted]

SEQ ID NO: 10 - protein, 126 aa, synthetic construct [sequence omitted]

SEQ ID NO: 11 - DNA, 642 bp, synthetic construct
  misc_feature 1..642: note = nirsevimab light chain
  source 1..642: mol_type = other DNA
gacatccagatgacccagtccccctcctctctgtctgctgccgtgggcgacagagtgaccatcacctgtcaggcctcccaggacatcgtgaactacctgaactggtatcagcagaagcccggcaaggcccccaagctgctgatctacgtggcctccaacctggaaaccggcgtgccctccagattctccggctctggctctggcaccgacttcagcctgaccatctccagcctgcagcctgaggacgtggccacctactactgccagcagtacgacaacctgcccctgacctttggcggaggcaccaaggtggagatcaaacgaactgtggctgcaccatctgtcttcatcttcccccccagcgacgagcagctgaagagcggcaccgcctccgtggtgtgcctgctgaacaacttctacccccgcgaggccaaggtgcagtggaaggtggacaacgccctgcagtccggcaacagccaggagagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaacaggggcgagtgc

SEQ ID NO: 12 - DNA, 1368 bp, synthetic construct
  misc_feature 1..1368: note = nirsevimab heavy chain
  source 1..1368: mol_type = other DNA
caagtgcagctggtgcagtctggcgccgaagtgaagaaacccggctcctccgtgatggtgtcctgccaggcttctggcggcctgctggaagattacatcatcaactgggtgcgacaggccccaggccagggacctgaatggatgggcggaatcatccccgtgctgggcaccgtgcactacggccctaagttccagggcagagtgaccatcaccgccgacgagtctaccgacaccgcctacatggaactgtcctccctgcggagcgaggacaccgccatgtactactgcgccaccgagacagccctggtggtgtccgagacatacctgccccactacttcgacaactggggccagggaaccctcgtgaccgtctcctcagcctccaccaagggcccatcggtcttccccctggcaccctcctccaagtccacctccggcggcaccgccgctctgggctgcctggtgaaggactacttccctgagcctgtgaccgtgtcctggaactctggcgccctgacctctggcgtgcacaccttccctgccgtgctgcagtcctccggcctgtactccctgtcctccgtggtgacagtgccttcctcctccctgggcacccagacctacatctgcaacgtgaaccacaagcccagcaacaccaaggtggacaagagagttgagcccaaatcttgtgacaaaactcacacatgcccaccgtgcccagcacctgaactcctggggggaccgtcagtctttctgttccctcctaagcctaaggacaccctgtacatcacccgggagcctgaagtgacctgcgtggtggtggatgtgtcccacgaggaccctgaggtgaagttcaattggtacgtggacggcgtggaggtgcacaacgccaagaccaagcctcgggaggagcagtacaactccacctaccgggtggtgtctgtgctgaccgtgctgcaccaggactggctgaacggcaaagaatacaagtgcaaagtctccaacaaggccctgcctgcccccatcgagaaaaccatctccaaagccaaagggcagccccgagaaccacaggtgtacaccctgcctccctcccgcgaggagatgaccaagaaccaggtgtccctgacctgtctggtgaagggcttctacccttccgatatcgccgtggagtgggagtccaacggccagcctgagaacaactacaagaccacccctcctgtgctggactccgacggctccttcttcctgtactccaagctgaccgtggacaagtcccggtggcagcagggcaacgtgttctcctgctccgtgatgcacgaggctctgcacaaccactacacccagaaaagcctctccctgtctccgggtaaa

SEQ ID NO: 13 - protein, 126 aa, synthetic construct [sequence omitted]

SEQ ID NO: 14 - protein, 126 aa, synthetic construct
  REGION 1..126: note = 1F5 heavy chain variable region
QVQLVQSGAEVKKPGSSVMVSCQASGGPLEDYIINWVRQAPGQGPEWMGGIIPVLGTVHYGPKFQGRVTITADESTDTAYMELSSLRSEDTAMYYCATETALVVSTTYLPHYFDNWGQGTLVTVSS

SEQ ID NO: 15 - protein, 126 aa, synthetic construct
  REGION 1..126: note = 2D10 heavy chain variable region
QVQLVQSGAEVKKPGSSVMVSCQASGGPLEDYIINWVRQAPGQGPEWMGGIIPVLGTVHYGPKFQGRVTITADESTDTAYMELSSLRSEDTAMYYCATETALVVSTTYRPHYFDNWGQGTLVTVSS

SEQ ID NO: 16 - protein, 126 aa, synthetic construct
  REGION 1..126: note = 1G7-GLM heavy chain variable region
QVQLVQSGAEVKKPGSSVKVSCKASGGLLEDYIINWVRQAPGQGPEWMGGIIPVLGTVHYGPKFQGRVTITADESTSTAYMHLSSLRSEDTAMYYCARETALVVSTTYLPHYFDNWGQGTLVTVSS

SEQ ID NO: 17 - protein, 126 aa, synthetic construct
  REGION 1..126: note = B12-1 heavy chain variable region
QVQLVQSGAEVKKPGSSVKVSCKASGGLLEDYIINWVRQAPGQGPEWMGGIIPVLGTVHYGPKFQGRVTITADESTDTAYMHLSSLRSEDTAMYYCATETALVVSTTYLPHYFDNWGQGTLVTVSS

SEQ ID NO: 18 - protein, 126 aa, synthetic construct
  REGION 1..126: note = E3-5 heavy chain variable region
QVQLVQSGAEVKKPGSSVKVSCKASGGLLEDYIINWVRQAPGQGPEWMGGIIPVLGTVHYGPKFQGRVTITADESTSTAYMELSSLRSEDTAMYYCATETALVVSTTYLPHYFDNWGQGTLVTVSS

SEQ ID NO: 19 - protein, 126 aa, synthetic construct
  REGION 1..126: note = E9-2 heavy chain variable region
QVQLVQSGAEVKKPGSSVKVSCKASGGLLEDYIINWVRQAPGQGPEWMGGIIPVLGTVHYGPKFQGRVTITADESTSTAYMHLSSLRSEDTAMYYCATETALVVSTTYLPHYFDNWGQGTLVTVSS